(19) United States
(12) Patent Application Publication
(10) Pub. No.: US 2024/0044882 A1
(43) Pub. Date: Feb. 8, 2024

(54) TETHERED DETECTION ASSAYS

(71) Applicant: Singular Genomics Systems, Inc., San Diego, CA (US)

(72) Inventor: Eli N. Glezer, Del Mar, CA (US)

(21) Appl. No.: 18/365,476

(22) Filed: Aug. 4, 2023

Related U.S. Application Data
(60) Provisional application No. 63/395,632, filed on Aug. 5, 2022.

(51) Int. Cl. G01N 33/543; G01N 33/58

(52) U.S. Cl. CPC G01N 33/54306; G01N 33/582

(57) ABSTRACT

Disclosed herein, inter alia, are compositions, complexes, and methods useful for efficient multiplex assays.

### CROSS-REFERENCES TO RELATED APPLICATIONS

This application claims the benefit of U.S. Provisional Application No. 63/395,632, filed Aug. 5, 2022, which is incorporated herein by reference in its entirety and for all purposes.

### SEQUENCE LISTING

The Sequence Listing written in file 051385-561001US_ST26.xml, created Jul. 28, 2023, 11,299 bytes, machine format IBM-PC, MS Windows operating system, is hereby incorporated by reference.

### BACKGROUND

Several methods have been developed that can detect very low concentrations of analyte molecules. For example, in sandwich enzyme-linked immunosorbent assay (ELISA), an immobilized antibody is used to capture an analyte followed by binding of a detectable, second antibody (i.e., forming a capture antibody-analyte-detection antibody sandwich). Sandwich immunoassay techniques provide highly sensitive and specificity for target analytes (e.g., antibodies, antigens, proteins, glycoproteins, and hormones) since two antibodies are used for capture (Ab_{C}) and detection (Ab_{D}). Despite some commercial successes, a major challenge is cross-reactivity (i.e., non-specificity) of detection antibodies binding to incorrect analytes, resulting in false positives, false negatives, and/or an increase in background noise. A significant amount of expensive reagents (e.g., Ab_{D}) is wasted when the target analyte is not captured from the sample. Disclosed herein, inter alia, are solutions to these and other problems in the art.

### BRIEF SUMMARY

In an aspect is provided a method of detecting a biomolecule in a sample, where the method includes contacting a solid support with a sample including a biomolecule, where the solid support includes a first biomolecule-specific binding agent attached to the solid support and a second biomolecule-specific binding agent attached to the solid support via a cleavable linker; forming a complex including the first biomolecule-specific binding agent bound to the biomolecule and the second biomolecule-specific binding agent bound to the biomolecule; cleaving the cleavable linker of the complex thereby forming a cleaved complex, wherein cleaving includes contacting the cleavable linker with a cleaving agent and detaching the second biomolecule-specific binding agent from the solid support; detecting the solid support; and detecting the cleaved complex. In embodiments, the biomolecule is a lipid, carbohydrate, peptide, protein, or antigen binding fragment.

In an aspect is provided a method of amplifying a polynucleotide sequence attached to a biomolecule-specific binding agent, the method including: contacting a solid support with a biomolecule, wherein the solid support includes: a first biomolecule-specific binding agent attached to the solid support; a second biomolecule-specific binding agent attached to the solid support via a cleavable linker, wherein the cleavable linker includes the polynucleotide sequence; forming a complex including an biomolecule bound to both the first biomolecule-specific binding agent bound and the second specific-binding agent; cleaving the cleavable linker of the complex thereby forming a cleaved complex; wherein cleaving includes contacting the cleavable linker with a cleaving agent and detaching the second biomolecule-specific binding agent from the solid support; and hybridizing a primer oligonucleotide to the polynucleotide sequence and extending the primer oligonucleotide sequence with a polymerase, thereby amplifying the polynucleotide sequence attached to the biomolecule-specific binding agent.

In an aspect is provided a solid support, including a first biomolecule-specific binding agent attached to the solid support; a second biomolecule-specific binding agent attached to the solid support via a cleavable linker, wherein the cleavable linker is a divalent linker including one or more cleavable sites, wherein the second biomolecule-specific binding agent includes a detectable moiety.

In an aspect is provided a plurality of particles wherein each particle is independently a particle as described herein and in related embodiments. In embodiments, each particle of the plurality includes a different first biomolecule-specific binding agent (e.g., a plurality of first biomolecule-specific binding agents).

### BRIEF DESCRIPTION OF THE DRAWINGS

FIGS. 1A-1B depict an embodiment of the solid support containing immobilized capture antibodies (Ab_{C}) and immobilized detection antibodies (Ab_{D}). The detection antibodies (indicated with a star) are immobilized to a solid support (e.g., a particle) via a cleavable tether, which optionally includes an oligonucleotide sequence. The cleavable tether is capable of being cleaved, thereby separating the tether from the substrate. FIG. 1B shows that following the capture of an appropriate biomolecule (e.g., analyte containing at least two antigenic epitopes capable of binding to Ab_{C} and Ab_{D}) by both the immobilized capture antibody (Ab_{C}) and the immobilized detection antibody (Ab_{D}), a complex is formed. Following the capture of the appropriate analyte, the cleavable tether is cleaved. Any unbound detection antibodies (i.e., detection antibodies that did not form a complex) are removed from the substrate following cleaving the cleavable tether. For multiplexing assays, multiple substrates will be simultaneously present (e.g., each substrate may be provided within a well of a multiwell container).

FIGS. 2A-2G illustrate the means for detecting the complex, and thus detecting the immobilized analyte. FIG. 2A provides an embodiment wherein following the cleavage of the cleavable tether containing a polynucleotide sequence, an extendable 3′ end is formed. This approach provides that the cleavage of the oligonucleotide sequence tethered to the Ab_{D} is required for subsequent signal generation. Thus, any residual un-cleaved tethers containing oligonucleotide sequences will not contribute a background signal. Subsequent detection of the Ab_{D} may then occur following hybridization and detection of a labeled linear oligonucleotide (FIG. 2B), wherein the linear oligonucleotide is already attached to a fluorophore and is detected, or one or more labeled nucleotides are incorporated by extending that 3′ end of the remnant of the tether (alternatively referred to herein as a cleaved complex) with labeled nucleotides (i.e., sequencing). Alternatively, a circularizable oligonucleotide may hybridize to the remnant, optional amplifying the circularizable oligonucleotide via rolling circle amplification (e.g., RCA or eRCA) with a DNA polymerase (depicted as a squishy-cloud object), followed by subsequent detection of the circularizable oligonucleotide (FIG. 2C). For example, as depicted in FIG. 2D, the cleaved portion of the tether oligonucleotide sequence serves as the primer for RCA. The circularizable oligonucleotide could either use a fully formed circle, or oligonucleotide capable of being ligated together to form a circle using the tether oligonucleotide sequence both as a splint and a primer. In embodiments, the circularizable oligonucleotide includes a padlock probe (PLP). In embodiments, generating an amplicon with multiple copies of the PLP greatly increases the limit of detection. As illustrated in FIG. 2E, the tether oligonucleotide sequence may include a unique molecular identifying (UMI or otherwise referred as identification oligonucleotide) sequence, on which the circularizable oligonucleotide probe anneals on the UMI, or on flanking positions to the UMI sequence. A polymerase (squishy-cloud object) extends the end of the circularizable oligonucleotide probe thereby incorporating a complement of the UMI into the circularizable oligonucleotide probe, as illustrated in FIG. 2F. The circularizable oligonucleotide probe may then be optionally amplified to form a polynucleotide containing many copies of the UMI sequence (FIG. 2G), which may then be detected.

FIG. 3. The methods and compositions described herein are useful for multiplexed assays. As illustrated in FIG. 3, a 96-well plate is provided, wherein each well includes different capture (Ab_{CN}) and detection antibody (Ab_{DN}) combinations. For example, Ab_{C1} antibodies are present in the same well (e.g., on the same particle within the same well) with the corresponding detection antibody Ab_{D1}. In embodiments, each well includes a capture antibody and detection antibody specific for a particular target biomolecule.

FIGS. 4A-4B illustrate embodiments for solid supports useful in the methods described herein. FIG. 4A provides a particle, optionally immobilized with a well with one or more immobilization oligonucleotides having complementary sequences to immobilized oligonucleotides attached to the well, having a plurality of capture antibodies immobilized to the particle and a plurality of detection antibodies immobilized to the particle via a cleavable tether, as described herein. The particle of FIG. 4A includes a substrate barcode, which contains an identifying sequence associated with the antibodies. FIG. 4B provides a particle including single or several chemiluminescent/fluorescent dyes to create a unique fluorescent signature (e.g., a Luminex® particle, or a spectrum signature particle). Each particle includes capture antibodies associated with the unique spectrum signature, thus enabling identification.

FIG. 5 provides an illustration of the cleavable linker as described herein. In embodiments, the covalent cleavable linker includes a first primer binding site (P1) and a second primer binding site (P2) providing binding sequences for a padlock probe (PLP). Shown in FIG. 5, the P1 and P2 sequences are immediately adjacent and PLP is circularized. However, it is understood that the two primer binding sequences may be separated by one or more nucleotides permitting a UMI sequence to be incorporated into the circle, as illustrated in FIG. 2F. The cleavable linker also includes one or more cleavable sites (CS), which are 3′ relative to the primer binding sites. The cleavable linker includes a bioconjugate linker to the detection antibody (Ab_{D}), which are upstream and downstream from the primer binding sites, respectively. In embodiments, the cleavable sites are separated from the primer binding sequences by one or more nucleotides.

### DETAILED DESCRIPTION

The aspects and embodiments described herein relate to tethered agent assays providing colocalized capture and detection agents.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a particle described herein to interact with an analyte.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” “binding,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the term “analyte” refers to a component, substance, or constituent of interest in an analytical procedure whose presence, absence, or amount is desired to be determined or measured. In an immunoassay, for example, the analyte may be a protein, protein fragment, polypeptide, an antibody, antibody fragment, antigen expressing antibody or a molecule detectable with an antibody, an antigen, an antigen binding fragment, a ligand, a lipid, a carbohydrate, or a derivative or combination thereof. The term “analyte” also refers to detectable components of structured elements such as cells, including all animal and plant cells, and microorganisms, such as fungi, viruses, bacteria including, but not limited to, all gram positive and gram negative bacteria, and protozoa.

In some embodiments, a “sample” includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

An “analyte-specific binding agent” or “biomolecule-specific binding agent” is a substance that allows for selective binding to another substance (e.g. an analyte). A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. A binding agent is typically a biological or synthetic molecule that has high affinity for another molecule or macromolecule, through covalent or non-covalent bonding. Examples of a binding agent can include streptavidin, antibody, antigen, enzyme, enzyme cofactor or inhibitor, hormone, or hormone receptor. This binding agent can bind to an analyte (e.g., a protein), often in a heterogeneous population of proteins and other biologics. Thus, under designated immunoassay conditions, the specified binding agents bind to a particular analyte at least two times the background and more typically more than 10 to 100 times background. In embodiments, a biomolecule-specific binding agent is attached to a solid support. In embodiments, a biomolecule-specific binding agent described herein is bound to a detectable moiety or agent (e.g., a fluorochrome). In embodiments, the biomolecule-specific binding agent can form a complex with an analyte and a second biomolecule-specific binding agent to facilitate detection of the analyte.

An “antibody” (Ab) is a protein that binds specifically to a particular substance, known as an “antigen” (Ag). An “antibody” or “antigen-binding fragment” is an immunoglobulin that binds a specific “epitope.” The term “antigen-binding site” of an antibody (or simply “antibody portion”), as used herein, refers to one or more fragments of an antibody that retains the ability to specifically bind to an antigen. It has been shown that the antigen-binding function of an antibody can be performed by fragments of a full-length antibody. The term “antibody” encompasses polyclonal, monoclonal, and chimeric antibodies. In nature, antibodies are generally produced by lymphocytes in response to immune challenge, such as by infection or immunization. An antibody may include the entire antibody as well as any antibody fragments capable of binding the antigen or antigenic fragment of interest. Examples of antibody functional fragments include, but are not limited to, include complete antibody molecules, antibody fragments, such as Fv, single chain Fv (scFv), complementarity determining regions (CDRs), VL (light chain variable region), VH (heavy chain variable region), fragment antigen binding (Fab), F(ab)2′ and any combination of those or any other functional portion of an immunoglobulin peptide capable of binding to target antigen (see, e.g., FUNDAMENTAL IMMUNOLOGY (Paul ed., 4th ed. 2001). Antibodies used herein are immunospecific for, and therefore specifically and selectively bind to, for example, proteins either detected (i.e., biological targets of interest) or used for detection (i.e., probes containing oligonucleotide barcodes) in the methods and devices as described herein.

A “monoclonal antibody” comprises a collection of identical molecules produced by a single B cell lymphocyte clone which are directed against a single antigenic determinant. Monoclonal antibodies can be distinguished from polyclonal antibodies in that monoclonal antibodies must be individually selected whereas polyclonal antibodies are selected in groups of more than one or, in other words, in bulk. Large amounts of monoclonal antibodies can be produced by immortalization of a polyclonal B cell population using hybridoma technology. Each immortalized B cell can divide, presumably indefinitely, and gives rise to a clonal population of cells that each expresses an identical antibody molecule. The individual immortalized B cell clones, the hybridomas, are segregated and cultured separately.

The term “polyclonal antibody” refers to an antibody that is produced from a different B cell lineages within the body. A polyclonal antibody is directed to many different antigenic determinants on the target cell surface and would bind with sufficient density to allow the effector mechanisms of the immune system to work efficiently.

An immunoglobulin (antibody) structural unit are typically tetrameric glycosylated proteins. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Thus, the term antibody, as used herein, includes antibody fragments either produced by the modification of whole antibodies, or those synthesized de novo either chemically or using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301. As appreciated by one of skill in the art, various antibody fragments can be obtained by a variety of methods, for example, digestion of an intact antibody with an enzyme, such as pepsin, or de novo synthesis.

The term “aptamer” refers to oligonucleotide or peptide molecules that bind to a specific target molecule. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In embodiments, peptide aptamers consist of one (or more) short variable peptide domains, attached at both ends to a protein scaffold. Aptamers may be designed with any combination of the base modified nucleotides desired. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can be identified using any known method, including the SELEX process. See, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues and organisms. At the molecular level, aptamers bind to its target site through non-covalent interactions. Aptamers bind to these specific targets because of electrostatic interactions, hydrophobic interactions, and their complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection.

An “antigen” (Ag) is any substance that reacts specifically with antibodies or T lymphocytes (T cells). In general, antigens include molecules or portions thereof that trigger an immune response in a host (e.g., in a subject), and may be recognized by an antibody. Antigens may be foreign to a subject (e.g., as in viral or bacterial proteins, polysaccharides, or other molecules), or native to the subject (e.g., as in an autoimmune response to self-proteins, which optionally may be mutant forms of a native protein). Examples of antigens include, without limitation, viral antigens, bacterial antigens, fungal antigens, cancer or tumor antigens, and allergens. Examples of viral antigens include, but are not limited to, env, gag, rev, tar, tat, nucleocapsid proteins and reverse transcriptase from immunodeficiency viruses (e.g., HIV, FIV), such as HIV-1 gag, HIV-1 env, HIV-1 pol, HIV-1 tat, HIV-1 nef; HBV surface antigen and core antigen, HbsAG, HbcAg; HCV antigens such as hepatitis C core antigen; influenza nucleocapsid proteins; parainfluenza nucleocapsid proteins; HPV E6 and E7 such as human papilloma type 16 E6 and E7 proteins; Epstein-Barr virus LMP-1, LMP-2 and EBNA-2; herpes LAA and glycoprotein D such as HSV glycoprotein D; as well as similar proteins from other viruses. In embodiments, the biomolecule-specific binding moiety is an antibody that is reactive to a plurality of viral antigens within the same viral group. For example, a flavivirus group-reactive antibody such as the monoclonal antibody MAb 6B6C-1, dengue 4G2, or Murray Valley 4A1B-9 is reactive with arbovirus antigens within the flavivirus genus, which includes the West Nile virus, Saint Louis encephalitis virus, Japanese encephalitis virus, and dengue virus. Similarly, for example, an alphavirus group-reactive antibody such as EEE 1A4B-6 or WEE 2A2C-3 is reactive with alphavirus antigens within the alphavirus genus, which includes eastern equine encephalitis virus, western equine encephalitis virus, and Venezuelan equine encephalitis virus. Similarly, for example, a bunyavirus group-reactive antibody such as LAC 10G5.4 is reactive with bunyavirus antigens within the bunyavirus genus, which includes the California serogroup of bunyaviruses, which includes La Crosse virus. Examples of bacterial antigens include, but are not limited, to capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or β-polysaccharide moieties of bacteria such as Pseudomonas aeruginosa serotype 011); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, Sbi, and von Willebrand factor binding protein. Non-limiting examples of fungal antigens include, but are not limited to, Candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. Examples of cancer antigens include, but are not limited to, MAGE, MART-1/Melan-A, gp100, dipeptidyl peptidase IV (DPPIV), adenosine deaminase-binding protein (ADAbp), cyclophilin b, colorectal associated antigen (CRC)-COI 7-1 A/GA733, carcinoembryonic antigen (CEA) and its immunogenic epitopes CAP-1 and CAP-2, etvβ, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, 1mp-1, P1 A, EBV-encoded nuclear antigen (EBNA)-I, brain glycogen phosphorylase, SSX-1, SSX-2 (HOM-MEL-40), SSX-1, SSX-4, SSX-5, SCP-1, CT-7, and c-erbB-2. Examples of allergens include, but are not limited to, dust, pollen, pet dander, food such as peanuts, nuts, shellfish, fish, wheat milk, eggs, soy and their derivatives, and sulphites. These lists are not meant to be limiting.

An “affimer” is a protein that binds to target proteins with affinity in the nanomolar range. It behaves similarly to an antibody by binding tightly to its target molecule. Affimers are recombinant proteins that are typically engineered to mimic molecular recognition characteristics of monoclonal antibodies.

As used herein, the term “immunoassay” refers to a biochemical test that measures the presence or concentration of a macromolecule or a small molecule in a solution involving a reaction between an antibody and an antigen. The molecule detected by the immunoassay is often referred to as an “analyte” and is in many cases a protein, although it may be other kinds of molecules, of different sizes and types. Immunoassays come in many different formats and variations. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogenous immunoassays or less frequently non-separation immunoassays. Immunoassays include assays in which the analyte is an antigen, as well as assays in which the analyte is an antibody (e.g., when detecting the presence, absence, or degree of an immune response). In embodiments, an immunoassay includes detecting multiple different analytes from a single sample simultaneously in a common reaction volume.

In an aspect provided herein, the biomolecule-specific binding agents use a cleavable linker to attach to the solid support. The use of a cleavable linker ensures that the label can, if required, be removed after detection, avoiding any interfering signal with any labelled nucleotide incorporated subsequently. The term “cleavable linker” can also be used interchangeably with the term “cleavable tether”. The use of the term “cleavable linker” is not meant to imply that the whole linker is required to be removed from the binding agent. The cleavage site can be located at a position on the linker that ensures that part of the linker remains attached to the binding agent after cleavage. The linker can be attached at any position on the binding agent.

The term “cleavable linker” or “cleavable tether” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. In embodiments, the cleavable linker is a covalent cleavable linker. In certain embodiments, the cleavable linker can include a polynucleotide or polypeptide sequence. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, made up of “dNTPs,” which do not have a hydroxyl group at the 2′ position, and oligoribonucleotides, made up of “NTPs,” which have a hydroxyl group in the 2′ position. Oligonucleotides also may include derivatives, in which the hydrogen of the hydroxyl group is replaced with an organic group, e.g., an allyl group.

Oligonucleotides, as described herein, typically are capable of forming hydrogen bonds with oligonucleotides having a complementary base sequence. These bases may include the natural bases, such as A, G, C, T, and U, as well as artificial, non-standard or non-natural nucleotides such as iso-cytosine and iso-guanine. As described herein, a first sequence of an oligonucleotide is described as being 100% complementary with a second sequence of an oligonucleotide when the consecutive bases of the first sequence (read 5′-to-3′) follow the Watson-Crick rule of base pairing as compared to the consecutive bases of the second sequence (read 3′-to-5′). An oligonucleotide may include nucleotide substitutions. For example, an artificial base may be used in place of a natural base such that the artificial base exhibits a specific interaction that is similar to the natural base.

An oligonucleotide that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of residue matches between at least two polynucleotide sequences aligned using a standardized algorithm (e.g., BLAST). An oligonucleotide that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (T m) for the specific sequence at a defined ionic strength and pH. The T m is the temperature (under defined ionic strength and pH) at which 50% of the target sequence hybridizes to a perfectly matched probe. Equations for calculating T, for example, nearest-neighbor parameters, and conditions for nucleic acid hybridization are known in the art.

As used herein, the term “identification oligonucleotide” can also refer to a “barcode” or “index” or “unique molecular identifier (UMI)” and refers to a known nucleic acid sequence which has feature(s) that can be identified. Typically, an identification oligonucleotide is unique to a particular feature in a pool of identification oligonucleotide that differ from one another in sequence, and each of which is associated with a different feature. In embodiments, identification oligonucleotides are about or at least about 5, 6, 7, 8, 9, 10, 15, 20, 25, 30, 40, 50, 75 or more nucleotides in length. In embodiments, identification oligonucleotides are shorter than 20, 15, 10, 9, 8, 7, 6, or 5 nucleotides in length. In embodiments, identification oligonucleotides are 10-50 nucleotides in length, such as 15-40 or 20-30 nucleotides in length. In a pool of different identification oligonucleotides, identification oligonucleotides may have the same or different lengths. In general, identification oligonucleotides are of sufficient length and comprise sequences that are sufficiently different to allow the identification of associated features (e.g., a binding agent or analyte) based on identification oligonucleotides with which they are associated. In embodiments, an identification oligonucleotide can be identified accurately after the mutation, insertion, or deletion of one or more nucleotides in the identification oligonucleotide sequence, such as the mutation, insertion, or deletion of 1, 2, 3, 4, 5, or more nucleotides. In embodiments, each identification oligonucleotide in a plurality of identification oligonucleotides differs from every other identification oligonucleotide in the plurality by at least three nucleotide positions, such as at least 3, 4, 5, 6, 7, 8, 9, 10, or more nucleotide positions. In embodiments, a barcode is a substrate barcode. In embodiments, a barcode is a substrate polynucleotide barcode. As used herein a “substrate barcode” is a barcode attached to a substrate or solid support (e.g., particle). As used herein, a “substrate polynucleotide barcode” is a barcode attached to a first biomolecule-specific binding moiety or analyte-specific capture antibody.

The terms “detect” and “detecting” as used herein refer to the act of viewing (e.g., imaging, indicating the presence of, quantifying, or measuring (e.g., spectroscopic measurement) an agent based on an identifiable characteristic of the agent, for example, the light emitted from detectable agents. In embodiments, the detectable agents could be conjugated to biomolecule-specific binding agents (e.g., antibodies). In embodiments, the solid support as described herein could have a composition containing detectable moieties (e.g., dyes or fluorochromes) in various ratios or concentrations that permit the detection of the solid support. Upon being exposed to an absorption light, the detectable agent will emit an emission light. The presence of an emission light can indicate the presence of the biomolecule-specific binding agents (and therefore, the bound target) or the solid support. Likewise, the quantification of the emitted light intensity can be used to measure the concentration of the agent.

As used herein, the term “detectable moiety” or “detectable agent” can also refer to a “label” or “labels” and generally refer to molecules that can directly or indirectly produce or result in a detectable signal either by themselves or upon interaction with another molecule. Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e. cyanine 3 or Cy3). In embodiments, the cyanine moiety has 5 methine structures (i.e. cyanine 5 or Cy5). In embodiments, the cyanine moiety has 7 methine structures (i.e., cyanine 7 or Cy7). A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the term “hybridize” or “specifically hybridize” refers to a process where two complementary nucleic acid strands anneal to each other under appropriately stringent conditions. Hybridizations are typically and preferably conducted with oligonucleotides. Non-limiting examples of nucleic acid hybridization techniques are described in, for example, Sambrook, et al., Molecular Cloning: A Laboratory Manual (2nd Edition, 1989). Those skilled in the art understand how to estimate and adjust the stringency of hybridization conditions such that sequences having at least a desired level of complementarity will stably hybridize, while those having lower complementarity will not. Hybridization reactions can be performed under conditions of different “stringency”. For example, a low stringency hybridization reaction is carried out at about 40° C. in 10×SSC. A moderate stringency hybridization may be performed at about 50° C. in 6×SSC. A high stringency hybridization reaction is generally performed at about 60° C. in 1×SSC. Hybridization reactions can also be performed under “physiological conditions” which is well known to one skilled in the art (e.g., a physiological condition is the temperature, ionic strength, pH and concentration of Mg′ normally found in vivo).

As used herein, the term “polymerase” is used in accordance with its plain ordinary meaning and refers to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-) A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A and E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L4085/Y409A/P410V mutations, NEB Therminator IX DNA polymerase), or γ-phosphate labeled nucleotides (e.g., Therminator γ: D141A/E143A/W355A/L408W/R460A/Q4615/K464E/D480V/R484W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entireties for all purposes.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which comprises a double stranded portion of nucleic acid.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction comprises a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that comprises a polymerase chain reaction (PCR). Conditions conducive to amplification (i.e., amplification conditions) are well known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers comprising tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification comprises a nucleic acid amplification reaction comprising only one species of oligonucleotide primer immobilized to a surface or substrate. In embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US 2013/0012399), or combinations thereof.

The methods and kits of the present disclosure may be applied, mutatis mutandis, to the sequencing of RNA, or to determining the identity of a ribonucleotide.

As used herein, the terms “sequencing”, “sequence determination”, and “determining a nucleotide sequence”, are used in accordance with their ordinary meaning in the art, and refer to determination of partial as well as full sequence information of the nucleic acid being sequenced, and particular physical processes for generating such sequence information. That is, the term includes sequence comparisons, fingerprinting, and like levels of information about a target nucleic acid, as well as the express identification and ordering of nucleotides in a target nucleic acid. The term also includes the determination of the identification, ordering, and locations of one, two, or three of the four types of nucleotides within a target nucleic acid. Sequencing produces one or more sequencing reads.

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to binding and/or incorporating one or more nucleotides (e.g., a compound described herein) to the 3′ end of a polynucleotide with a polymerase, and detecting one or more labels that identify the one or more nucleotides. The sequencing may be accomplished by, for example, sequencing by synthesis, sequencing by binding, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate | Bioconjugate | |
|---|---|---|
| reactive group 1 | reactive group 2 | |
| (e.g., electrophilic | (e.g., nucleophilic | Resulting |
| bioconjugate | bioconjugate | Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or |
| diazoalkanes | carboxylic acids | anhydrides |
| epoxides | thiols | esters |
| haloacetamides | thiols | thioethers |
| haloplatinate | amino | thioethers |
| haloplatinate | heterocycle | platinum complex |
| haloplatinate | thiol | platinum complex |
| halotriazines | amines/anilines | platinum complex |
| halotriazines | alcohols/phenols | aminotriazines |
| halotriazines | thiols | triazinyl ethers |
| imido esters | amines/anilines | triazinyl |
| isocyanates | amines/anilines | thioethers |
| isocyanates | alcohols/phenols | amidines |
| isothiocyanates | amines/anilines | ureas |
| maleimides | thiols | urethanes |
| phosphoramidites | alcohols | thioureas |
| silyl halides | alcohols | thioethers |
| sulfonate esters | amines/anilines | phosphite esters |
| sulfonate esters | thiols | silyl ethers |
| sulfonate esters | carboxylic acids | alkyl amines |
| sulfonate esters | alcohols | thioethers |
| sulfonyl halides | amines/anilines | esters |
| sulfonyl halides | phenols/alcohols | ethers |
| | | sulfonamides |
| | | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group

“\*MERGEFORMAT\*MERGEFORMAT (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, or halogen bond) or van der Waals interactions (e.g., dipole-dipole, dipole-induced dipole, or London dispersion). In embodiments, the non-covalent linker is the result of two molecules that are not covalently linked to each other that interact with each other via a non-covalent bond.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “particle” means a small body made of a rigid or semi-rigid material. The body can have a shape characterized, for example, as a sphere, oval, microsphere, or other recognized particle shape whether having regular or irregular dimensions. The particles may in one way or another rest upon a two dimensional surface by magnetic, gravitational, or ionic forces, or by chemical bonding, or by any other means known to those skilled in the art. In further embodiments, the bead may have magnetic properties. Further the beads may have a density that allows them to rest upon a two dimensional surface in solution. Particles may consist of glass, polystyrene, latex, metal, quantum dot, polymers, silica, metal oxides, ceramics, or any other substance suitable for binding to nucleic acids, or chemicals or proteins which can then attach to nucleic acids. The particles may be rod shaped or spherical or disc shaped, or comprise any other shape. The particles may also be distinguishable by their shape or size or physical location. The particles may be distinguished through spectroscopy by having a composition containing dyes or fluorochromes in various ratios or concentrations. The particles may also be distinguishable by barcode or holographic images or other imprinted forms of particle coding. Where the particles are magnetic particles, they may be attracted to the surface of the chamber by application of a magnetic field and the magnetic particles may be dispersed from the surface of the chamber by removal of the magnetic field. The magnetic particles are preferably paramagnetic or superparamagnetic.

The term “gel” in this context refers to a semi-rigid solid that is permeable to liquids and gases. As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. Hydrogels can contain over 99% water and may comprise natural or synthetic polymers, or a combination thereof. Hydrogels also possess a degree of flexibility very similar to natural tissue, due to their significant water content. A detailed description of suitable hydrogels may be found in published U.S. Patent Application 2010/0055733, herein specifically incorporated by reference. By “hydrogel subunits” or “hydrogel precursors” is meant hydrophilic monomers, prepolymers, or polymers that can be crosslinked, or “polymerized”, to form a three-dimensional (3D) hydrogel network. Hydrogels can be derived from a single species of monomer or from two or more different monomer species with at least one hydrophilic component. Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO-PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel includes one or more crosslinks including hydrogen bonds, hydrophobic interactions, and/or polymer chain entanglements.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. The term “kit” includes both fragmented and combined kits. In embodiments, the kit includes, without limitation, nucleic acid primers, nucleotides, probes, adapters, enzymes, buffers, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, peptides, proteins, antigen binding fragments, antibodies, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

Described herein are complexes including a first biomolecule-specific binding agent bound to a biomolecule and a second biomolecule-specific binding agent bound to the biomolecule, wherein the first biomolecule-specific binding agent is attached to a solid support, and wherein the second biomolecule-specific binding agent is attached to the solid support via a cleavable linker. Prior to cleaving the cleavable linker, the complex is attached to the solid support via both the first and the second biomolecule-specific binding agents. Following cleavage of the cleavable linker, the complex may be referred to as a “cleaved complex”, wherein the cleaved complex is attached to the solid support via the first biomolecule-specific binding agent. For example, the cleaved complex includes the first biomolecule-specific binding agent bound to the biomolecule and the second biomolecule-specific binding agent bound to the same biomolecule, wherein the cleaved complex is attached to the solid support via the first biomolecule-specific binding agent. The cleaved complex may include a remnant of the cleaved linker attached to the second biomolecule-specific binding agent, e.g., a portion of the cleavable linker may remain attached to the second biomolecule-specific binding agent following cleavage.

The abbreviations used herein have their conventional meaning within the chemical and biological arts. The chemical structures and formulae set forth herein are constructed according to the standard rules of chemical valency known in the chemical arts.

### II. Compositions, Devices, & Kits

Provided herein in an aspect is a solid support, including a first biomolecule-specific binding agent attached to the solid support; a second biomolecule-specific binding agent attached to the solid support via a cleavable linker, wherein the cleavable linker is a divalent linker including one or more cleavable sites, wherein the second biomolecule-specific binding agent includes a detectable moiety. In embodiments, the detectable moiety is an oligonucleotide including a fluorophore. In embodiments, the divalent linker is a covalent linker including one or more cleavable sites. In embodiments, the divalent linker is a covalent linker including a plurality of cleavable sites. In embodiments, the divalent linker is directly attached to the solid support and does not include hybridizing any portion of the linker to an immobilized oligonucleotide.

In an aspect is provided a solid support, which includes a first analyte-specific binding agent attached to the solid support; a second analyte-specific binding agent attached to the solid support via a cleavable linker, wherein the cleavable linker is a divalent linker includes one or more cleavable sites, wherein the second analyte-specific binding agent includes a detectable moiety. In embodiments, an analyte-specific binding agent is a biomolecule-specific binding agent.

In embodiments, the solid support further includes one or more oligonucleotides, wherein the one or more oligonucleotides are attached to the solid support. The solid support may be decorated with bioconjugate reactive moieties (e.g., where the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety) so that one or more oligonucleotide moieties may be bound to the solid support through the bioconjugate reactive moieties. In some embodiments, the oligonucleotides moiety is about 5 to about 25 nucleotides in length. In embodiments, the oligonucleotide moiety is a substrate barcode, and includes a known sequence associated with the first biomolecule-specific binding agent. The oligonucleotides may serve to immobilize the solid support to a second solid support (e.g., wherein the particle includes one or more immobilization oligonucleotides complementary to immobilized oligonucleotides in a well).

In embodiments, the solid support is a particle. In embodiments, a solid support may be a particle, such as a bead. In embodiments, the substrate is a paramagnetic bead. In embodiments, the substrate is a superparamagnetic bead. Paramagnetic and superparamagnetic particles have negligible magnetism in the absence of a magnetic field. The application of a magnetic field induces alignment of the magnetic domains in the particles, which results in attraction of the particles to the field source. When the field is removed, the magnetic domains return to a random orientation so there is no interparticle magnetic attraction or repulsion. In the case of superparamagnetism, this return to random orientation of the domains is nearly instantaneous, while paramagnetic materials will retain domain alignment for some period of time after removal of the magnetic field. If the particles have a sufficient density, they may be attracted to the bottom surface of the chamber by gravity and dispersed from the bottom surface of the chamber by agitation of the chamber, such as by vortexing, sonication, or fluidic movement. Agitation of the chamber may also be used to further assist in dispersing particles in methods and systems in which the particles were attracted to a surface of the chamber by other forces, such as magnetic or ionic forces, or suction forces, or vacuum filtration, or affinity, or hydrophilicity or hydrophobicity, or any combination thereof.

In embodiments, the particle is a hydrogel particle. In embodiments, the hydrogel particle is comprised of agarose- and acrylamide-based gels. In embodiments, the hydrogel particle is comprised of polyacrylamide, poly-N-isopropylacrylamide, poly N-isopropylpolyacrylamide, 2-hydroxyethyl acrylate and methacrylate, zwitterionic monomers, polyethylene glycol acrylate and methacrylate. In embodiments, the hydrogel particle is comprised of acrylic acid. In embodiments, the particle includes a hydrogel (e.g., the particle is coated in a hydrogel).

In embodiments, the particle includes glass, ceramic, metal, silica, magnetic material, or a paramagnetic material. The particle may be an inorganic particle. The inorganic particle may be a metal particle. When the particle is a metal, the metal may be titanium, zirconium, gold, silver, platinum, cerium, arsenic, iron, aluminum, or silicon. The metal particle may be titanium, zirconium, gold, silver, or platinum and appropriate metal oxides thereof. In embodiments, the particle is titanium oxide, zirconium oxide, cerium oxide, arsenic oxide, iron oxide, aluminum oxide, or silicon oxide. The metal oxide particle may be titanium oxide or zirconium oxide. The particle may be titanium. The particle may be gold. The particle may be silicon dioxide. The particle may be silica. The particle may include a silica core and polymer shell. In embodiments, the particle is a streptavidin particle. In embodiments, the particle includes streptavidin.

In embodiments, the particle is a polymer particle includes polymerized units of polyacrylamide (AAm), poly-N-isopropylacrylamide, poly N-isopropylpolyacrylamide, sulfobetaine acrylate (SBA), carboxybetaine acrylate (CBA), phosphorylcholine acrylate (PCA), sulfobetaine methacrylate (SBMA), carboxybetaine methacrylate (CBMA), phosphorylcholine methacrylate (PCMA), polyethylene glycol acrylate, methacrylate, polyethylene glycol (PEG)-thiol/PEG-acrylate, acrylamide/N,N′-bis(acryloyl)cystamine (BACy), PEG/polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, glicydyl methacrylate (GMA), hydroxyethyl methacrylate (HEMA), hydroxyethyl acrylate (HEA), hydroxypropyl methacrylate (HPMA), polyethylene glycol methacrylate (PEGMA), polyethylene glycol acrylate (PEGA), isocyanatoethyl methacrylate (IEM), or a copolymer thereof.

In embodiments, the average longest dimension of the particle is from about 100 nm to about 3000 nm. In embodiments, the average longest dimension of the particle is from about 1000 nm to about 3000 nm. In embodiments, the average longest dimension of the particle is from about 500 nm to about 2000 nm. In embodiments, the average longest dimension of the particle is from 100 to 900 nm. In embodiments, the average longest dimension of the particle is from 1000 to 1900 nm. In embodiments, the average longest dimension of the particle is from 2000 to 3000 nm. In embodiments, the average longest dimension of the particle is from 200 to 700 nm. In embodiments, the average longest dimension of the particle is from 250 to 650 nm. In embodiments, the average longest dimension of the particle is from 300 to 600 nm. In embodiments, the average longest dimension of the particle is from 350 to 550 nm. In embodiments, the average longest dimension of the particle is from 400 to 500 nm. In embodiments, the average longest dimension of the particle is from 450 to 550 nm. In embodiments, the average longest dimension of the particle is from 500 to 600 nm. In embodiments, the average longest dimension of the particle is 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, or 650 nm.

In embodiments, the solid support is a particle including two or more fluorescent dyes within or on the particle. For example, the particle may be a multicolored, fluorescently stained small particles of generally less than 100 μm in diameter (e.g., approximately 10 nm to 100 μm in diameter), wherein the ratio of fluorescent dyes creates a unique spectrum signature for each particle that may be known and thus associated with the capture antibody. The specific ratio or proportion of dyes within a population of particles will determine the location of said populations on a fluorescence map of a multiwell container (e.g., an array), which allocates these populations according to fluorescent color and brightness. By using as little as two dyes, e.g., orange and red, as many as 64 populations of beads are made each one distinct from another by subtle variations in unique fluorescence characteristics recognized by standard detection devices (e.g., flow cytometry instrument). In embodiments, the solid support is a polymeric microsphere (e.g., polystyrene or latex). Any type of polymeric material of microspheres is acceptable including but not limited to brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, or combinations thereof. The particles may also include 1 to 30% of a cross-linking agent, such as divinyl benzene, ethylene glycol dimethacrylate, trimethylolpropane trimethacrylate, or N,N′methylene-bis-acrylamide or other functionally equivalent agents known in the art. In embodiments, the particles are made of polystyrene and contain 1 to 30% divinyl benzene. See for example, the particles and methods of making particles as described in U.S. Pat. Nos. 6,632,526 and/or 8,568,881, each of which are incorporated herein in their entireties.

In embodiments, the solid support is a particle including a plurality of dyes (e.g., two or more dyes). In embodiments, the two or more fluorescent dyes are selected from cyclobutenedione derivatives, symmetrical and unsymmetrical squaraines, substituted cephalosporin compounds, fluorinated squaraine compositions, alkylalkoxy squaraines, or squarylium compounds. Squarylium compounds or squaraines as used herein refer to fluorescent compounds derived from squaric acid containing a cyclobutene moiety flanked between two aromatic moieties. In embodiments, the two or more fluorescent dyes are selected from 1,3-bis[(1,3-dihydro-1,3,3-trimethyl-2H-indol-2-ylidene)methyl]-2,4-dihydroxycyclobutenediylium, bis(inner salt) and 2-(3,5-dimethylpyrrol-2-yl)-4-(3,5-dimethyl-2H-pyrrol-2-ylidene)-3-hydroxy-2-cyclobuten-1-one. The molar ratio between first and second dye, when present in a particle, may be between about 0 and 10,000, more preferably between 0.00001 and 2,000. Both dyes would preferably be excited at the same absorption wavelength, e.g., ranging from ultraviolet to about 800 nm, and emit fluorescent light at two spectrally distinct wavelengths, which are spectrally resolved by at least 10 nm, preferably 30 nm, and more preferably by at least 50 nm. For example, the emission peak of the dye #1 is at 585 nm, and the peak emission of dye #2 is at 630 nm.

In embodiments, the solid support is a particle including polystyrene, brominated polystyrene, polyacrylic acid, polyacrylonitrile, polyacrylamide, polyacrolein, polydimethylsiloxane, polybutadiene, polyisoprene, polyurethane, polyvinyl acetate, polyvinylchloride, polyvinylpyridine, polyvinylbenzylchloride, polyvinyltoluene, polyvinylidene chloride, polydivindylbenzene, polyglycidylmethacrylate, polymethylmethacrylate, or copolymers, blends, composites, or combination thereof.

In embodiments, the particle is in a well of a multiwell container. In embodiments, the container includes a plurality of wells, wherein one or more wells include a particle. In embodiments, there is at least one particle per well. In embodiments, there is at most one particle per well. In embodiments, the particles are non-covalently attached to the wells. In embodiments, the particles are physiosorbed to the wells. Particles may be loaded into wells through several methods known in the art. For example, particles loading may simply be gravity driven. Gravity driven loading may also be accelerated by subsequently spinning down the array in a centrifuge, or with an orbital mixer to increase the particle settling rate. Such combinations are optimized so that no more than one particle is loaded into a given well to achieve near complete coverage of the array with high uniformity. In other embodiments, sonication and/or physical wiping with a flat tool may be used as a post-loading cleaning technique to reduce doubly-loaded wells and clear interstitial regions of particles. Post-cleaning may also simply consist of rinsing with a solvent to remove non-specifically bound particles.

In embodiments, the particle includes one or more fluorescent labels. In embodiments, the fluorescent label is a fluorescent molecule. In further embodiments, the fluorescent label is an acridine dye, cyanine dye, fluorine dye, oxazine dye, phenanthridine dye, rhodamine dye, or squarylium dye. In embodiments, the fluorescent dye is capable of exchanging energy with another fluorescent dye (e.g., fluorescence resonance energy transfer (FRET) chromophores). In embodiments, the fluorescent label is commercially available CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.).

In embodiments, the first biomolecule-specific binding agent and the second biomolecule-specific binding agent are each an agent capable of selectively binding a protein. In embodiments, the first biomolecule-specific binding agent and the second biomolecule-specific binding agent each specifically bind a particular protein (e.g., protein antigen or epitope). In embodiments, the first biomolecule-specific binding agent and the second biomolecule-specific binding agent are each an agent capable of selectively binding a lipid. In embodiments, the first biomolecule-specific binding agent and the second biomolecule-specific binding agent are each an agent capable of selectively binding a carbohydrate. In embodiments, the first biomolecule-specific binding agent and the second biomolecule-specific binding agent are each an agent capable of selectively binding a peptide. In embodiments, the first biomolecule-specific binding agent and the second biomolecule-specific binding agent are each an agent capable of selectively binding an antigen binding fragment. In embodiments, the first biomolecule-specific binding agent and the second biomolecule-specific binding agent are each an agent capable of selectively binding an antibody. In embodiments, the first biomolecule-specific binding agent and the second biomolecule-specific binding agent are an immunoglobulin. In embodiments, the first biomolecule-specific binding agent is an immunoglobulin. In embodiments, the second biomolecule-specific binding agent is an immunoglobulin. In embodiments, the immunoglobulin is IgA, IgD, IgE, IgG, or IgM. In embodiments, the immunoglobulin is IgA. In embodiments, the immunoglobulin is IgD. In embodiments, the immunoglobulin is IgE. In embodiments, the immunoglobulin is IgG. In embodiments, the immunoglobulin is IgM.

In embodiments, the first biomolecule-specific binding agent and the second biomolecule-specific binding agent are each an antibody. In embodiments, the first biomolecule-specific binding agent is an antibody. In embodiments, the second biomolecule-specific binding agent is an antibody. In embodiments, specific binding to an antibody would occur under conditions that utilize an antibody that is selected for its specificity for a particular biomolecule. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular biomolecule.

In embodiments, the first biomolecule-specific binding agent and the second biomolecule-specific binding agent are each an antibody, single-chain Fv fragment (scFV), antibody fragment-antigen binding (Fab), affimer, or an aptamer. In embodiments, the first biomolecule-specific binding agent is an antibody, single-chain Fv fragment (scFV), antibody fragment-antigen binding (Fab), affimer, or an aptamer. In embodiments, the second biomolecule-specific binding agent is an antibody, single-chain Fv fragment (scFV), antibody fragment-antigen binding (Fab), affimer, or an aptamer. In embodiments, the first biomolecule-specific binding agent is a single-chain Fv fragment (scFV), antibody fragment-antigen binding (Fab), affimer, or an aptamer. In embodiments, the second biomolecule-specific binding agent is a single-chain Fv fragment (scFV), antibody fragment-antigen binding (Fab), affimer, or an aptamer. In embodiments, the first biomolecule-specific binding agent is a single-chain Fv fragment (scFV). In embodiments, the first biomolecule-specific binding agent is an antibody fragment-antigen binding (Fab). In embodiments, the first biomolecule-specific binding agent is an affimer. In embodiments, the first biomolecule-specific binding agent is an aptamer. In embodiments, the second biomolecule-specific binding agent is a single-chain Fv fragment (scFV). In embodiments, the second biomolecule-specific binding agent is an antibody fragment-antigen binding (Fab). In embodiments, the second biomolecule-specific binding agent is an affimer. In embodiments, the second biomolecule-specific binding agent is an aptamer.

In embodiments, the first biomolecule-specific binding agent and the second biomolecule-specific binding agent are each an antigen-specific antibody. In embodiments the first biomolecule-specific binding agent is an antigen-specific antibody. In embodiments the second biomolecule-specific binding agent is an antigen-specific antibody. In embodiments, the first biomolecule-specific binding agent and the second biomolecule-specific binding agent are each the antigen-binding site (e.g., fragment antigen-binding (Fab) variable region) of an antibody. In embodiments, the antigen-specific antibody is an intact antibody. In embodiments, the intact antibody is a Fab fragment, F(ab′)2 fragment, an Fd fragment, an Fv fragment, a dAb fragment and an isolated CDR. In embodiments, the intact antibody is a Fab fragment. In embodiments, the intact antibody is an F(ab′)2 fragment. In embodiments, the intact antibody is an Fd fragment. In embodiments, the intact antibody is an Fv fragment. In embodiments, the intact antibody is a dAb fragment. In embodiments, the intact antibody is an isolated CDR. In embodiments, the Fab fragment is a monovalent fragment consisting of the VL, VH, CL and CH1 domains. In embodiments, the F(ab′)2 fragment is a bivalent fragment comprising two Fab fragments linked by a disulfide bridge at the hinge region. In embodiments, the Fd fragment consists of the VH and CH1 domains. In embodiments, the Fv fragment consists of the VL and VH domains of a single arm of an antibody. In embodiments, the dAb fragment consists of a VH domain. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). A commonly used linker is a 15-residue (Gly 4 Ser) 3 peptide, but other linkers are also known in the art.

In embodiments, the first biomolecule-specific binding agent and the second biomolecule-specific binding agent are each a monoclonal antibody or polyclonal antibody. In embodiments, the first biomolecule-specific binding agent is a monoclonal antibody. In embodiments, the first biomolecule-specific binding agent is a polyclonal antibody. In embodiments, the second biomolecule-specific binding agent is a monoclonal antibody. In embodiments, the second biomolecule-specific binding agent is a polyclonal antibody. In embodiments, the first biomolecule-specific binding agent is a monoclonal antibody, and the second biomolecule-specific binding agent is a polyclonal antibody. In embodiments, the first biomolecule-specific binding agent is a polyclonal antibody and the second biomolecule-specific binding agent is a monoclonal antibody.

In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

In an aspect is provided a plurality of particles wherein each particle is independently a particle as described herein and in related embodiments. In embodiments, each particle of the plurality includes a different first biomolecule-specific binding agent (e.g., a plurality of first biomolecule-specific binding agents).

In embodiments, one or more particles of the plurality is in a well of a multiwell container. In embodiments, the well is about 3 mm in diameter. In embodiments, the well is about 3.6 mm in diameter. In embodiments, the well is about 4 mm in diameter. In embodiments, the well is about 5 mm in diameter. In embodiments, the well is about 6 mm in diameter. In embodiments, the well is about 6.5 mm in diameter. In embodiments, the well is about 7 mm in diameter. In embodiments, the well is about 7.5 mm in diameter. In embodiments, the well is about 8 mm in diameter. In embodiments, the well is 5 mm in diameter. In embodiments, the well is 6 mm in diameter. In embodiments, the well is 6.5 mm in diameter. In embodiments, the well is 7 mm in diameter. In embodiments, the well is 7.5 mm in diameter. In embodiments, the well is 8 mm in diameter. In embodiments, the well is about 6 to 12 mm in depth. It is also understood that the size of the wells of the multiwell container can be of various sizes and will ultimately depend on the systems and/or apparatus used to analyze later reactions.

In embodiments, the wells of the multiwell container are separated from each other by about 1 mm to about 10 mm. In embodiments, the wells of the multiwell container are separated from each other by about 2 mm. In embodiments, the wells of the multiwell container are separated from each other by about 3 mm. In embodiments, the wells of the multiwell container are separated from each other by about 4 mm. In embodiments, the wells of the multiwell container are separated from each other by about 5 mm. In embodiments, the wells of the multiwell container are separated from each other by about 6 mm. In embodiments, the wells of the multiwell container are separated from each other by about 7 mm. In embodiments, the wells of the multiwell container are separated from each other by about 8 mm. In embodiments, the wells of the multiwell container are separated from each other by about 9 mm. It is also understood that the separation of the wells of the multiwell container will ultimately depend on the systems and/or apparatus used to analyze later reactions.

In embodiments, the wells of the multiwell container are separated from each other by about 0.2 μm to about 2.0 μm. In embodiments, the wells of the multiwell container are separated from each other by about 0.3 μm. In embodiments, the wells of the multiwell container are separated from each other by about 0.4 μm. In embodiments, the wells of the multiwell container are separated from each other by about 0.5 μm. In embodiments, the wells of the multiwell container are separated from each other by about 0.6 μm. In embodiments, the wells of the multiwell container are separated from each other by about 0.7 μm. In embodiments, the wells of the multiwell container are separated from each other by about 0.8 μm. In embodiments, the wells of the multiwell container are separated from each other by about 0.9 μm. In embodiments, the wells of the multiwell container are separated from each other by about 1.0 μm. In embodiments, the wells of the multiwell container are separated from each other by about 1.1 μm. In embodiments, the wells of the multiwell container are separated from each other by about 1.2 μm. In embodiments, the wells of the multiwell container are separated from each other by about 1.3 μm. In embodiments, the wells of the multiwell container are separated from each other by about 1.4 μm. In embodiments, the wells of the multiwell container are separated from each other by about 1.5 μm. In embodiments, the wells of the multiwell container are separated from each other by about 1.6 μm. In embodiments, the wells of the multiwell container are separated from each other by about 1.7 μm. In embodiments, the wells of the multiwell container are separated from each other by about 1.8 μm. In embodiments, the wells of the multiwell container are separated from each other by about 1.9 μm. It is also understood that the separation of the wells of the multiwell container will ultimately depend on the systems and/or apparatus used to analyze later reactions. In embodiments, each particle is bound to a discrete site on a substrate, wherein each discrete site of the substrate is separated by an interstitial region.

In embodiments, the well contains a gel. In embodiments, the gel has a colloidal structure. In embodiments the gel is agarose; polymer mesh structure, such as gelatin; or cross-linked polymer structure, such as polyacrylamide or a derivative thereof. In embodiments, analytes, such as polynucleotides, can be attached to the gel via covalent or non-covalent means. Exemplary methods and reactants for attaching nucleic acids to gels are described, for example, in US 2011/0059865, which is incorporated herein by reference. In embodiments the analytes, sample, tissue, or cell can include nucleic acids and the nucleic acids can attach to the gel or polymer via their 3′ oxygen, 5′ oxygen, or at other locations along their length such as via a base moiety of the 3′ terminal nucleotide, a base moiety of the 5′ nucleotide, and/or one or more base moieties elsewhere in the molecule.

The wells may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern. A regular array of such regions is advantageous for detection and data analysis of signals collected from the arrays during an analysis.

In embodiments, the well is substantially square. In embodiments, the well is square. In embodiments, the well is F-bottom. In embodiments, the wells are substantially round flat bottom wells. In embodiments, the well is C-bottom. In embodiments, the well is V-bottom. In embodiments, the well is U-bottom.

In embodiments, the multiwell container includes 24 substantially round flat bottom wells. In embodiments, the multiwell container includes 48 substantially round flat bottom wells. In embodiments, the multiwell container includes 96 substantially round flat bottom wells. In embodiments, the multiwell container includes 384 substantially round flat bottom wells. In embodiments, the multiwell container includes 24 substantially square flat bottom wells. In embodiments, the multiwell container includes 48 substantially square flat bottom wells. In embodiments, the multiwell container includes 96 substantially square flat bottom wells. In embodiments, the multiwell container includes 384 substantially square flat bottom wells.

In embodiments, each particle is bound to a discrete site on a substrate (e.g., each particle includes immobilization oligonucleotides and is non-covalently bound to complementary immobilized oligonucleotides), wherein each discrete site of the substrate is separated by an interstitial region. An interstitial region can separate one concave feature of an array from another concave feature of the array. The two regions that are separated from each other can be discrete, lacking contact with each other. In another example, an interstitial region can separate a first portion of a feature from a second portion of a feature. In embodiments, the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer). In embodiments, each particle of the plurality includes a different first biomolecule-specific binding agent as described herein and in related embodiments. In embodiments, two or more wells comprise different particles, wherein each particle includes a different first biomolecule-specific binding agent as described herein and in related embodiments.

In an aspect is provided a microfluidic device. In embodiments, the microfluidic device includes the plurality of particles wherein each particle is independently a particle as described herein and in related embodiments. In embodiments the microfluidic device includes an imaging system. In embodiments, the microfluidic device includes one or more reaction vessels or solid support where reagents interact and are imaged. Exemplary systems having fluidic components that can be readily modified for use in a system herein include, but are not limited to, those set forth in U.S. Pat. Nos. 8,241,573, 8,039,817; or US Pat. App. Pub. No. 2012/0270305 A1, each of which are incorporated herein by reference. In embodiments, the microfluidic device further includes one or more excitation lasers.

In embodiments, the microfluidic device is a nucleic acid sequencing device. Nucleic acid sequencing devices utilize excitation beams to excite labeled nucleotides in the DNA containing sample to enable analysis of the base pairs present within the DNA. Many of the next-generation sequencing (NGS) technologies use a form of sequencing by synthesis (SBS), wherein modified nucleotides are used along with an enzyme to read the sequence of DNA templates in a controlled manner. In embodiments, sequencing includes a sequencing by synthesis event, where individual nucleotides are identified iteratively (e.g., incorporated and detected into a growing complementary strand), as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444, and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. In embodiments, the nucleic acid sequencing device utilizes the detection of four different nucleotides that comprise four different labels.

In an aspect is provided a kit. In embodiments, the kit includes a composition as described herein. In embodiments, the kit includes the reagents and containers useful for performing the methods as described herein. Generally, the kit includes one or more containers providing a composition and one or more additional reagents (e.g., a buffer suitable for polynucleotide extension and/or sequencing). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores).

In as aspect is provided a kit including the proximity probe (i.e., biomolecule-specific binding agent) and oligonucleotide primer of any one of the aspects and embodiments herein.

In embodiments, the kit includes a microplate, and reagents for sample preparation and purification, amplification, and/or sequencing (e.g., one or more sequencing reaction mixtures). In embodiments, the kit includes for protein detection includes a plurality of proximity probes (i.e., biomolecule-specific binding agents) linked to an oligonucleotide (e.g., DNA-conjugated antibodies).

In embodiments, amplification reagents and other reagents may be provided in lyophilized form. In embodiments, amplification reagents and other reagents may be provided in a container that includes wells within which the lyophilized reagent may be reconstituted.

In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit includes a sequencing polymerase and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

### III. Methods

In an aspect is provided a method of detecting a biomolecule in a sample. In embodiments, the method includes contacting a solid support with a sample including a biomolecule, and the solid support includes a first biomolecule-specific binding agent attached to the solid support; and a second biomolecule-specific binding agent attached to the solid support via a cleavable linker; forming a complex including the first biomolecule-specific binding agent bound to the biomolecule and the second biomolecule-specific binding agent bound to the biomolecule; cleaving the cleavable linker of the complex thereby forming a cleaved complex, wherein cleaving includes contacting the cleavable linker with a cleaving agent and detaching the second biomolecule-specific binding agent from the solid support; detecting the solid support; and detecting the cleaved complex. In embodiments, the biomolecule is a lipid, carbohydrate, peptide, protein, or antigen binding fragment.

In an aspect is provided a method of detecting an analyte, the method includes contacting a solid support with an analyte, wherein the solid support includes a first analyte-specific binding agent attached to the solid support; and a second analyte-specific binding agent attached to the solid support via a cleavable linker thereby forming a complex that includes the analyte bound to both the first analyte-specific binding agent and the second analyte-specific binding agent; cleaving the cleavable linker of the complex thereby forming a cleaved complex; wherein cleaving includes contacting the cleavable linker with a cleaving agent and detaching the second analyte-specific binding agent from the solid support; and detecting the cleaved complex, thereby detecting the analyte.

In an aspect is provided a method of detecting a biomolecule in a sample, the method including contacting a solid support with a sample including a biomolecule (e.g., a lipid, carbohydrate, peptide, protein, or antigen binding fragment) and the solid support includes a first biomolecule-specific binding agent attached to the solid support (e.g., via a covalent, non-cleavable linker); and a second biomolecule-specific binding agent attached to the solid support via a covalent cleavable linker, thereby forming a complex comprising the first biomolecule-specific binding agent bound to the biomolecule and the second biomolecule-specific binding agent bound to the biomolecule; cleaving the cleavable linker of the complex thereby forming a cleaved complex; wherein cleaving comprises contacting the cleavable linker with a cleaving agent and detaching the second biomolecule-specific binding agent from the solid support; and detecting the cleaved complex. In embodiments, the covalent cleavable linker does not hybridize to a complementary immobilize oligonucleotide. In embodiments, the covalent cleavable linker does not include a sequence capable of hybridizing to an immobilized oligonucleotide.

In embodiments, the analyte or biomolecule is detected from a sample. A sample (e.g., a sample comprising an analyte) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, parasite nucleic acid).

In embodiments, the analyte, alternatively referred to herein as a biomolecule, may be a peptide, protein, or glycoprotein. In embodiments, the analyte is an amino acid, carbohydrate, nucleic acid, lipid, or toxin. In embodiments, the analyte is a steroid. In embodiments, the analyte is a vitamin. In embodiments, the analyte is a virus or virus particles. Analytes to be detected also include, but are not limited to, neurotransmitters, hormones, growth factors, antineoplastic agents, cytokines, monokines, lymphokines, nutrients, enzymes, receptors, antibacterial agents, antiviral agents and antifungal agents, and combinations thereof. In embodiments, the analyte is a molecule (e.g., organic or inorganic molecule). In embodiments, the analyte will have at least one epitope that an antibody or a binding agent can recognize. In embodiments, the biomolecule is a lipid, carbohydrate, peptide, protein, or antigen binding fragment. In embodiments, the biomolecule is a lipid. In embodiments, the biomolecule is a carbohydrate. In embodiments, the biomolecule is a peptide. In embodiments, the biomolecule is a protein. In embodiments, the biomolecule is antigen binding fragment.

In embodiments, the first biomolecule-specific binding agent is an antibody, single-chain Fv fragment (scFv), antibody fragment-antigen binding (Fab), affimer, or an aptamer. In embodiments, the first biomolecule-specific binding agent is an antibody. In embodiments, the first biomolecule-specific binding agent is a single-chain Fv fragment (scFv). In embodiments, the first biomolecule-specific binding agent is an antibody fragment-antigen binding (Fab). In embodiments, the first biomolecule-specific binding agent is an affirmer. In embodiments, the first biomolecule-specific binding agent is an aptamer.

In embodiments, the second biomolecule-specific binding agent is an antibody, single-chain Fv fragment (scFv), antibody fragment-antigen binding (Fab), affimer, or an aptamer. In embodiments, the second biomolecule-specific binding agent is an antibody. In embodiments, the second biomolecule-specific binding agent is a single-chain Fv fragment (scFv). In embodiments, the second biomolecule-specific binding agent is an antibody fragment-antigen binding (Fab). In embodiments, the second biomolecule-specific binding agent is an affirmer. In embodiments, the second biomolecule-specific binding agent is an aptamer. In embodiments, the second biomolecule-specific binding agent includes a detectable moiety.

In embodiments, the first biomolecule-specific binding agent and the second biomolecule-specific binding agent are both an antibody. In embodiments, the first biomolecule-specific binding agent is a monoclonal antibody and the second biomolecule-specific binding agent is a polyclonal antibody. In embodiments, the first biomolecule-specific binding agent and the second biomolecule-specific binding agent are present at different ratios. For example, the ratio of the first biomolecule-specific binding agent (e.g., the capture antibody) and the second biomolecule-specific binding agent (e.g., the detection antibody) is 0.75 to 1.0. In embodiments, the ratio of the first biomolecule-specific binding agent (e.g., the capture antibody) and the second biomolecule-specific binding agent (e.g., the detection antibody) is 0.16 to 1.0. In embodiments, the ratio of the first biomolecule-specific binding agent (e.g., the capture antibody) and the second biomolecule-specific binding agent (e.g., the detection antibody) is 6.0 to 10.0. In embodiments, the total number of first biomolecule-specific binding agents is greater than the total number of second biomolecule-specific binding agents on a particle. For example, a particle including 0.1 μM of detection antibodies and 0.6 μM of capture antibodies may be preferable.

In embodiments, the first biomolecule-specific binding agent and the second biomolecule-specific binding agent are independently a receptor or a ligand-binding portion thereof. In general, receptors include proteins that transmit a signal in a signaling pathway in response to binding a ligand. Receptors may be intracellular receptors or cell surface receptors. Examples of cell surface receptors include ligand-gated ion channels, G protein-coupled receptors, and receptor tyrosine kinases. Examples of receptors include, without limitation, tyrosine kinase receptor, such as a colony stimulating factor 1 (CSF-1), platelet-derived growth factor (PDGF), epidermal growth factor (EGF), transforming growth factor (TGF), nerve growth factor (NGF), insulin, insulin-like growth factor 1 (IGF-1) receptor, etc.; a G-protein coupled receptor, such as a Gi-coupled, Gq-coupled or Gs-coupled receptor, e.g. a muscarinic receptor (e.g. the subtypes m1, m2, m3, m4, m5), dopamine receptor (e.g. the subtypes D1, D2, D4, D5), opiate receptor (e.g. the subtypes μ or δ), adrenergic receptor (e.g. the subtypes α1A, α1B, α1C, α2C10, α2C2, α2C4), serotonin receptor, tachykinin receptor, luteinising hormone receptor or thyroid-stimulating hormone receptor, retinoic acid/steroid super family of receptors, mutant forms of receptors such as mutant TrkA receptor, mutant EGF receptors, ligand-gated channels including subtypes of nicotinic acetylcholine receptors, GABA receptors, glutamate receptors (NMDA or other subtypes), subtype 3 of the serotonin receptor, and the cAMP-regulated channel. In embodiments, the first biomolecule-specific binding agent and the second biomolecule-specific binding agent are each a ligand. In general, ligands include proteins that bind to and alter the function of a protein (e.g., an enzyme or a receptor). Ligands may be other proteins, protein fragments, or other molecules. Non-limiting examples of ligands include peptides, polypeptides or proteins, such as cytokines or growth factors. For example, ligands include but are not limited to βc, Cyclophilin A, IL-1, IL-2, IL-3, IL-4, IL-5, IL-6, IL-7, IL-8, IL-10, IL-12, IL-13, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, MCP1, MCP2, KC, MCP5, MCP4, MCP5, M-CSF, MIP1, MIP2, NOF, NT 3, NT4, NT5, NT6, NT7, OSM, PBP, PBSF, PDGF, PECAM-1, PF4, RANTES, SCF, TGF-α, TGF-β₁, TGF-β₂, TGF-β₃, TNF-α, TNF-β, TPO, VEGF, GH, chemokines, and eotaxin (eotaxin-1, -2 or -3).

In embodiments, the solid support includes a first biomolecule-specific binding agent attached to the solid support (e.g., a particle). In embodiments, the solid support includes a first biomolecule-specific binding agent attached to the solid support via a linker or a spacer. In embodiments, the solid support includes a first biomolecule-specific binding agent attached to the solid support via a non-cleavable linker. The first biomolecule-specific binding agent may be attached to the solid support through passive adsorption. In embodiments, the solid support is pre-coated with proteins that are attached using glutathione, metal-chelate, or other means. In embodiments, the solid support can have a polymer layer to increase passive adsorption.

In embodiments, the solid support comprises a first biomolecule-specific binding agent attached to the solid support via a linker (e.g., a non-cleavable covalent linker). In embodiments, the first biomolecule-specific binding agent is immobilized on the substrate via a first linker (e.g., a covalent linker) and the second biomolecule-specific binding agent is immobilized to the substrate via a second linker (e.g., a cleavable covalent linker), wherein the second linker includes one or more cleavable sites. The linkers may also include spacers. Including spacers in the linker puts the biomolecule-specific binding agent in an environment having a greater resemblance to free solution. This can be beneficial, for example, in enzyme-mediated reactions. It is believed that such reactions suffer less steric hindrance issues that can occur when the biomolecule-specific binding agent is directly attached to the solid support or is attached through a very short linker (e.g., a linker comprising about 1 to 3 carbon atoms). Attachment can be achieved via a bioconjugate reactive moiety present at the end of the linker, for example an azide moiety, a dibenzocyclooctyne (DBCO) moiety, or any other bioconjugate reactive moiety. The linker may be a carbon-containing chain such as those of formula —(CH₂)ₙ— wherein “n” is from 1 to about 1000. In embodiments, the linker includes polyethylene glycol (PEG) spacers having a general formula of —(CH₂—CH₂—O)ₘ—, wherein m is from about 1 to 500, 1 to 100, or 1 to 12. In embodiments, m is 3, 4, 5, 6, 7, 8, 9, 10, 11, or 12. In embodiments, the first biomolecule-specific agent is attached to the solid support via a bioconjugate linker, for example an NHS-PEG4-biotin, NHS-PEG12-biotin, ChromaLink®, or Sulfo-NHS-LC-biotin linker.

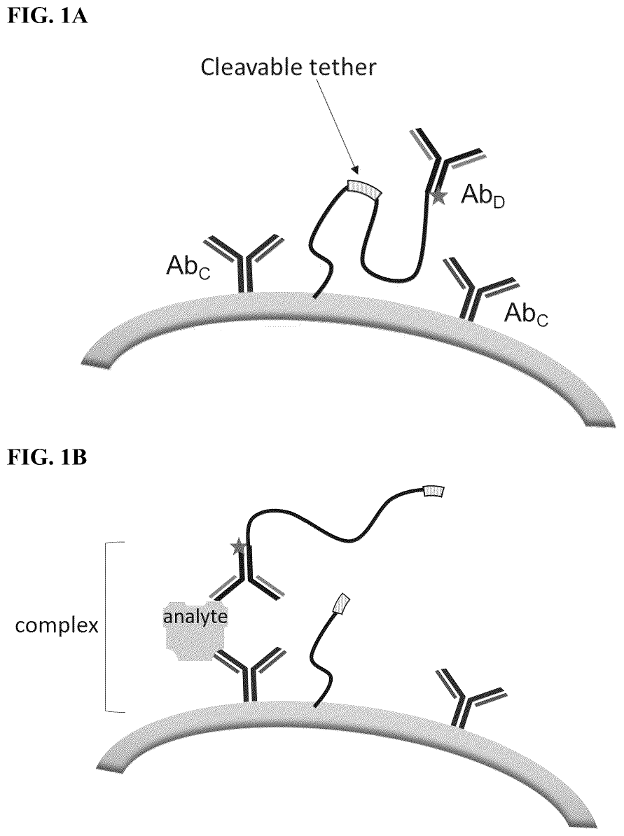

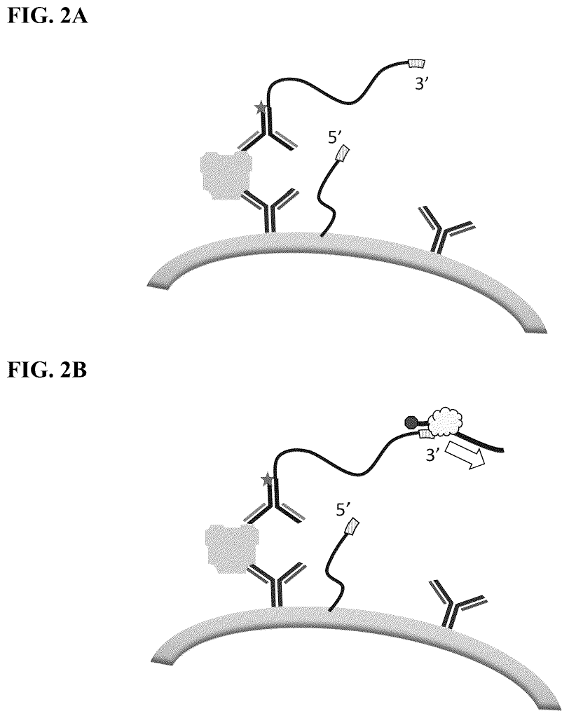

In embodiments, the biomolecule is bound to both the first biomolecule-specific binding agent and second biomolecule-specific binding agent thereby forming a complex. In embodiments, the complex comprises a biomolecule bound to a first biomolecule-specific binding agent attached to a solid support and the same biomolecule is bound to a second biomolecule-specific binding agent, which is attached to the solid support via a cleavable linker as shown in FIG. 1B. In embodiments, the biomolecule may be bound to the first biomolecule-specific agent through non-covalent or covalent means (e.g., following complex formation, first biomolecule-specific agent may be crosslinked to the biomolecule). In embodiments, the biomolecule may be bound to the second biomolecule-specific agent through non-covalent or covalent means (e.g., following complex formation, second biomolecule-specific agent may be crosslinked to the biomolecule). In embodiments, the first biomolecule-specific binding moiety and the second biomolecule-specific binding are cross-linked. Crosslinking is the process of joining two or more molecules, such as by a covalent bond, non-covalent interactions, or interactions with one or more intermediate molecules. Examples of crosslinking reagents (or crosslinkers) include molecules that contain two or more reactive ends capable of chemically attaching to specific functional groups (e.g., primary amines, sulfhydryls, etc.) on proteins or other molecules. The crosslinking may be direct, for example, via a covalent bond. The cross-linking may be indirect, for example, by forming a complex of antibodies that join the first biomolecule-specific binding agent to the second biomolecule-specific binding agent (which may optionally be stabilized by further cross-linking). By way of example, the complex of antibodies may include anti-mouse antibody binding moieties and/or Fc antibodies. Various cross-linking reagents and processes are available, particularly for cross-linking proteins. Examples of common crosslinkers include dimethyl suberimidate (contains imidoester), BS3 (contains N-Hydroxysuccinimide-ester), and formaldehyde. Each of these crosslinkers undergo nucleophilic attack from the amino group of lysine and subsequent covalent bonding via the crosslinker. The zero-length carbodiimide crosslinker EDC functions by converting carboxyls into amine-reactive isourea intermediates that bind to lysine residues or other available primary amines. SMCC or its water-soluble analog, Sulfo-SMCC, is commonly used to prepare antibody-hapten conjugates for antibody development. An in vitro cross-linking method, termed PICUP (photo-induced cross-linking of unmodified proteins) is a process in which ammonium persulfate (APS), which acts as an electron acceptor, and tris-bipyridylruthenium (II) cation ([Ru(bpy)₃]²⁺) are added to the protein of interest and irradiated with UV light. In vivo crosslinking of protein complexes using photo-reactive amino acid analogs is a method in which cells are grown with photoreactive diazirine analogs to leucine and methionine, which are incorporated into proteins. Upon exposure to ultraviolet light, the diazirines are activated and bind to interacting proteins that are within a few Angstroms of the photo-reactive amino acid analog (UV cross-linking).

In embodiments, the cleavable linker of the complex is cleaved thereby forming a cleaved complex. Following cleavage of the cleavable linker, the complex may be referred to as a “cleaved complex”, wherein the cleaved complex is attached to the solid support via the first biomolecule-specific binding agent. For example, the cleaved complex includes the first biomolecule-specific binding agent bound to the biomolecule and the second biomolecule-specific binding agent bound to the same biomolecule, wherein the cleaved complex is attached to the solid support via the first biomolecule-specific binding agent. In embodiments, the cleaved complex refers to the remnant of the cleaved linker attached to the second biomolecule-specific binding agent. In embodiments, cleaving the cleavable linker causes the second biomolecule-specific binding agent to detach from the solid surface as shown in FIG. 1B. In embodiments, cleaving the cleavable linker includes contacting the cleavable linker with a cleaving agent. In embodiments, the cleavable linker includes a polynucleotide or a polypeptide sequence. In embodiments, the cleavable linker includes a polynucleotide sequence. In embodiments, the covalent cleavable linker is a polynucleotide sequence attached to the solid support via a first bioconjugate linker and is attached to the second biomolecule-specific binding agent via a second bioconjugate linker. In embodiments, the cleavable linker does not include a hybridization sequence to retain the second biomolecule-specific agent to the solid support (e.g., the cleavable linker does not hybridize to an immobilized oligonucleotide to attach the second biomolecule-specific agent to the solid support).

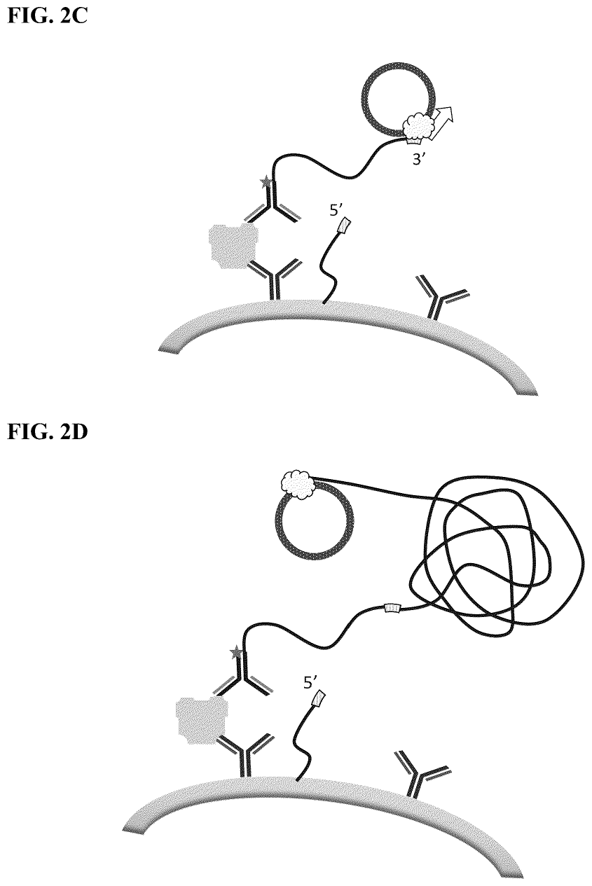

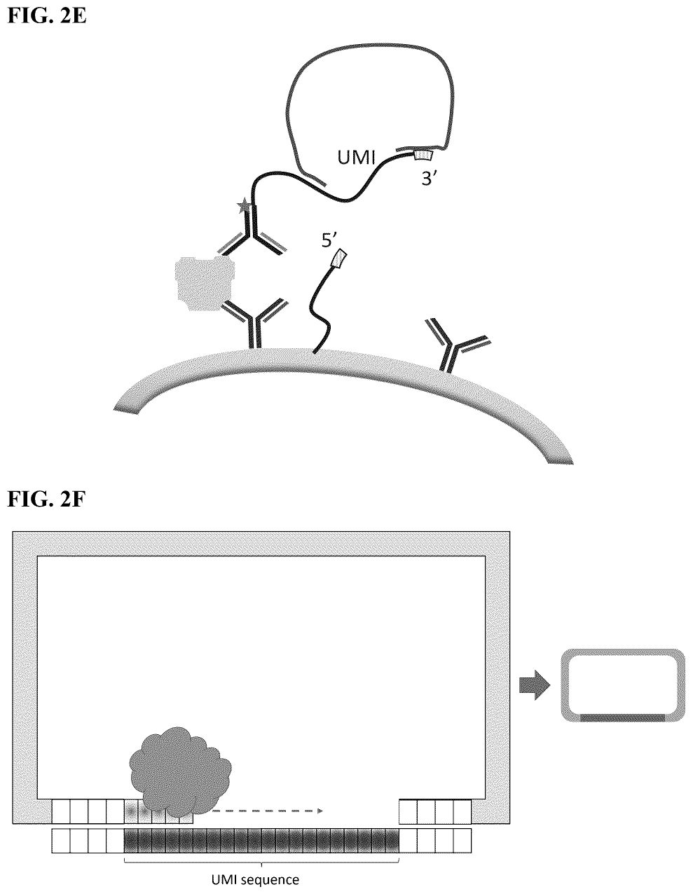

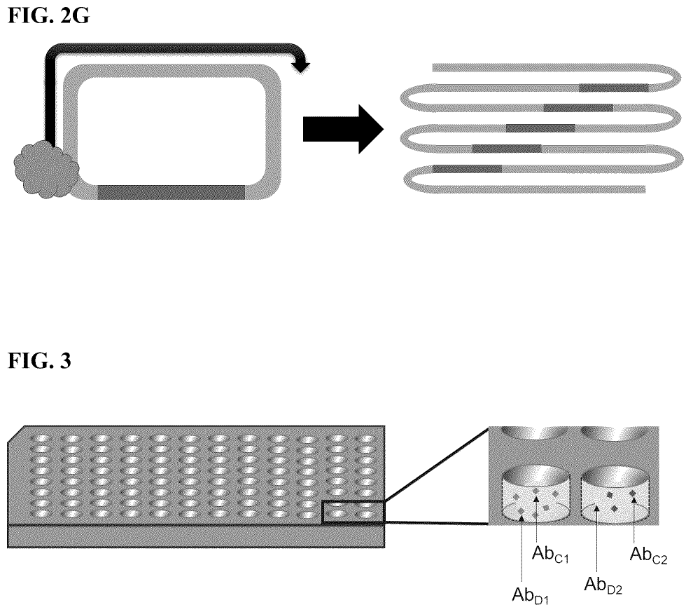

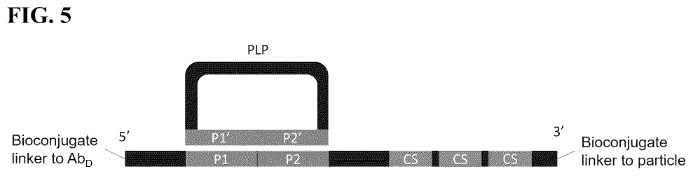

In embodiments, the cleavable linker includes a polynucleotide sequence. In embodiments, cleaving the cleavable linker includes generating an identification oligonucleotide, wherein the identification oligonucleotide includes a portion of the polynucleotide sequence. In embodiments, the method further includes amplifying the identification oligonucleotide (i.e., UMI sequence) to generate amplification products including multiple copies of the identification oligonucleotide, or a complement thereof as depicted in FIGS. 2E-2F. For example, as illustrated in FIG. 5, the covalent cleavable linker includes a first primer binding site (P1) and a second primer binding site (P2) providing binding sequences for a padlock probe (PLP). In embodiments, the cleavable linker includes one or more phosphorothioate moieties (e.g., in the primer binding sequence). Shown in FIG. 5, the P1 and P2 sequences are immediately adjacent and PLP is circularized, however it is understood that the two primer binding sequences may be separated by one or more nucleotides permitting a UMI sequence to be incorporated into the circle, as illustrated in FIG. 2F. The cleavable linker also includes one or more cleavable sites (CS), which are 3′ to the primer binding sites. In embodiments, the cleavable sites are separated from the primer binding sequences by one or more nucleotides. In embodiments, the PLP is allowed to contact the cleaved complex following cleavage, wherein a ligase circularizes the PLP to form a circular polynucleotide.

In embodiments, the cleavable linker is divalent moiety capable of being separated into distinct entities, where one entity remains attached to the solid support and the other entity (e.g., the cleaved complex) remains attached to the second biomolecule-specific agent. In embodiments, the cleavable linker is a monovalent moiety, which is capable of being separated into distinct entities. A cleavable linker is specifically cleavable in response to external stimuli. In embodiments, the cleavable linker is cleaved by contacting the cleavable linker with a cleaving agent. In embodiments, the cleavable linker can be cleaved by enzymes, nucleophilic/basic reagents, reducing agents, photo-irradiation, electrophilic/acidic reagents, organometallic and metal reagents, or oxidizing reagents. In embodiments, the cleavable linker can be chemically cleaved by a chemical. In embodiments, the chemically cleavable linker is split in response to the presence of a acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄). In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, a chemically cleavable linker is non-enzymatically cleavable. In embodiments, cleaving includes removing. In embodiments, the cleavable linker includes one or more cleavable site(s). Any suitable enzymatic, chemical, or photochemical cleavage reaction may be used to cleave the cleavable site. In embodiments, cleaving the cleavable linker can be chemical cleavage of a bond between a deoxyribonucleotide and a ribonucleotide, in which case the cleavable site may include one or more ribonucleotides. In embodiments, cleaving of the cleavable site can be a chemical reduction of a disulfide linkage with a reducing agent (e.g., THPP or TCEP), in which case the cleavable site should include an appropriate disulfide linkage; chemical cleavage of a diol linkage with periodate, in which case the cleavable site should include a diol linkage; generation of an abasic site and subsequent hydrolysis, etc. In embodiments, the linker includes a diol linkage, which permits cleavage by treatment with periodate (e.g., sodium periodate). It will be appreciated that more than one diol can be included at the cleavable site. One or more diol units may be incorporated into a polynucleotide using standard methods for automated chemical DNA synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, the cleavable linker comprises two or more cleavable sites. Any suitable enzymatic, chemical, or photochemical cleavage reaction may be used to cleave the cleavable site. In embodiments, the cleavable site includes one or more deoxyuracil nucleobases (dUs). In embodiments, the cleavable site includes multiple deoxyuracil nucleobases (dUs). In embodiments, the cleavable site includes a plurality of consecutive nucleobases (dUs). In embodiments, the cleavable site is cleaved as a result of enzymatic cleaving. In embodiments, the cleaving agent is an enzyme. In embodiments, the enzyme is one or more restriction enzymes. The restriction enzyme will recognize a particular restriction site sequences in one or both strands of the cleavable site, resulting in cleavage of the cleavable site. The resulting restriction enzyme digestion may cleave one or both strands of a duplex template. The enzymatic cleavage reaction may result in removal of a part or the whole of the strand being cleaved. In embodiments, the restriction enzyme recognition sequence included in the cleavable site is selected to be a “rare-cutting” restriction enzyme recognition sequence, e.g., a restriction enzyme that cuts with low frequency in any given genome. For example, Nod is a rare cutter with an eight-base recognition site, which will occur on average about once every 65,000 base pairs in a genome (assuming an average frequency of each type of canonical base of ¼). Other rare-cutting enzymes are known in the art and commercially available, including AbsI, AscI, BbvCI, CciNI, FseI, MreI, PaIAI, RigI, SdaI, and SgsI.

In embodiments, the cleaving agent includes a reducing agent, sodium periodate, or a nuclease. In embodiments, the cleaving agent includes RNase, Formamidopyrimidine DNA Glycosylase (Fpg), a restriction enzyme, or uracil DNA glycosylase (UDG), or a combination thereof. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents. In embodiments, agents that split cleavable sites at or near the modified nucleotide or bond are collectively referred herein as “cleaving agents.” Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme, such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example, a type IIS restriction endonuclease. In embodiments, the cleaving agent is an exonuclease (e.g., RecBCD), restriction nuclease, endoribonuclease, exoribonuclease, or RNase (e.g., RNAse I, II, or III). In embodiments, the cleaving agent is a restriction enzyme. In embodiments, the cleaving agent includes a glycosylase and one or more suitable endonucleases. In embodiments, cleavage of the cleavable site(s) is performed under alkaline (e.g., pH greater than 8) buffer conditions at between 40° C. to 80° C.

In embodiments, the cleavable linker includes a polynucleotide or a polypeptide sequence. In embodiments, the cleavable linker includes a polynucleotide sequence. In embodiments, cleaving the cleavable linker includes generating an identification oligonucleotide, wherein the identification oligonucleotide includes a portion of the polynucleotide sequence. In embodiments, the identification oligonucleotides are part of a polynucleotide or polypeptide sequence attached to the solid support. In embodiments the polynucleotide or polypeptide sequence is part of a cleavable linker attached to the solid support. In embodiments, the polynucleotide sequence is an identification oligonucleotide also known as a barcode. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. An oligonucleotide barcode is at least about 1, 2, 3, 4, 5, 6, 7, 8, 9, 10, 15, 20, 25, 30, 35, 40, 45, 50 or more nucleotides in length. An oligonucleotide barcode can be at most about 300, 200, 100, 90, 80, 70, 60, 50, 40, 30, 20, 15, 12, 10, 9, 8, 7, 6, 5, 4 or fewer or more nucleotides in length. In embodiments, an oligonucleotide barcode includes between about 5 to about 8, about 5 to about 10, about 5 to about 15, about 5 to about 20, about 10 to about 150 nucleotides. In embodiments, an oligonucleotide barcode includes between 5 to 8, 5 to 10, 5 to 15, 5 to 20, 10 to 150 nucleotides. In embodiments, an oligonucleotide barcode is 10 nucleotides.

An oligonucleotide barcode (i.e., identification oligonucleotide) may include a unique sequence (e.g., a barcode sequence or a unique molecule identifying (UMI) sequence) that provides the oligonucleotide barcode its identifying functionality. The unique sequence may be random or non-random. Attachment of the oligonucleotide barcode sequence to a biomolecule of interest (i.e., the target) may associate the barcode sequence with the biomolecule of interest. The barcode may then be used to identify the biomolecule of interest during sequencing, even when other biomolecules of interest (e.g., comprising different oligonucleotide barcodes) are present. In embodiments, the oligonucleotide barcode consists only of a unique barcode sequence. In embodiments, the 5′ end of a barcoded oligonucleotide is phosphorylated. In embodiments, the oligonucleotide barcode is known (i.e., the nucleic sequence is known before sequencing) and is sorted into a basis-set according to their Hamming distance. Oligonucleotide barcodes can be associated with a biomolecule of interest by knowing, a priori, the biomolecule of interest, such as a gene or protein. In embodiments, the oligonucleotide barcodes further include one or more sequences capable of specifically binding a gene or biomolecule of interest. For example, in embodiments, the oligonucleotide barcode includes a sequence capable of hybridizing to mRNA, e.g., one containing a poly-T sequence (e.g., having several T's in a row, e.g., 4, 5, 6, 7, 8, or more T's).

In embodiments, a barcode (i.e., identification oligonucleotide) is a degenerate or partially-degenerate sequence, such that one or more nucleotides are selected at random from a set of two or more different nucleotides at one or more positions, with each of the different nucleotides selected at one or more positions represented in a pool of oligonucleotides comprising the degenerate or partially-degenerate sequence. The number of possible barcodes in a given set of barcodes will vary with the number of degenerate positions, and the number of bases permitted at each such position. For example, a barcode of five nucleotides (consecutive or non-consecutive), in which each position can be any of A, T, G, or C represents 4⁵, or 1024 possible barcodes. In embodiments, certain barcode sequences may be excluded from a pool, such as barcodes in which every position is the same base. In embodiments, there are about, 10², 10³ 10⁴, 10⁵, 10⁶, 10⁷, 10⁸, 10⁹, or a number or a range between any two of these values, unique nucleotide barcode sequences. In embodiments, there are at least, or at most 10², 10³ 10⁴, 10⁵, 10⁶, 10⁷, 10⁸, 10⁹ unique barcode sequences. In embodiments, a barcode is about, 1, 2, 3, 4, 5, 10, 15, 20, 25, 30, 35, 40, 45, 50, or a number or a range between any two of these values, nucleotides in length. A barcode can be at least, or at most, 1, 2, 3, 4, 5, 10, 15, 20, 25, 30, 35, 40, 45, 50, 100, or 200 nucleotides in length. In embodiments, such as where the number of barcodes exceeds the number of first and/or second protein-binding moieties, a diverse set of barcode sequences are attached to a given species. In some embodiments, a diverse set of barcode sequences are attached to a given first biomolecule-specific binding moiety, or a substrate attached thereto.

In embodiments, the barcode (i.e., identification oligonucleotide) is a substrate polynucleotide barcode, for example, a polynucleotide barcode attached to a substrate or a first biomolecule-specific binding moiety. In embodiments, the barcode is a substrate polynucleotide barcode attached to a substrate. In embodiments, the solid support includes a substrate polynucleotide barcode. In embodiments, the barcode is a substrate polynucleotide barcode attached to a first biomolecule-specific binding moiety. In embodiments, the first biomolecule-specific binding moiety includes a substrate polynucleotide barcode. In embodiments, the substrate polynucleotide barcode is attached to the substrate using known techniques in the art. In embodiments, the substrate polynucleotide barcode is attached to the first biomolecule-specific binding moiety using known techniques in the art. In embodiments, the barcode is a protein polynucleotide barcode, for example, a polynucleotide barcode attached to a second biomolecule-specific binding moiety. In embodiments, none of the substrate polynucleotide barcodes are the same as any of the protein polynucleotide barcodes. In other embodiments, a set of substrate polynucleotide barcodes is permitted to have barcode sequences in common with a set of protein polynucleotide barcodes. For example, both the substrate polynucleotide barcodes and the protein polynucleotide barcodes may include a random 4-mer sequence, such that one or more barcode associated with a particular first biomolecule-specific binding moiety may also be associated with a particular second biomolecule-specific binding moiety. In cases where a one or more barcodes are associated with both a first and second biomolecule-specific binding moiety, the barcode sets may be sequenced in separate sequencing reactions (e.g., by including a different primer binding sequence for substrate polynucleotide barcodes as compared to a primer binding sequence for protein polynucleotide barcodes), so as to distinguish first biomolecule-specific binding moieties from second biomolecule-specific binding moieties based on the barcode sequences. For example, a sequencing process with a first primer can be used to sequence substrate polynucleotide barcodes, followed by another sequencing process with a second primer used to sequence protein polynucleotide barcodes, or vice versa.

Barcodes can be of any of a variety of lengths. Substrate polynucleotide barcodes may or may not all be the same length. Protein polynucleotide barcodes may or may not all be the same length. Substrate polynucleotide barcodes may or may not be the same length as protein polynucleotide barcodes. In embodiments, the substrate polynucleotide barcode, the protein polynucleotide barcode, or both are 1-50, 5-45, 10-40, 15-35, or 20-30 nucleotides in length. In embodiments, the substrate polynucleotide barcode, the protein polynucleotide barcode, or both are 10-50, 15-45, 20-40, or 25-35 nucleotides in length. In embodiments, the substrate polynucleotide barcode, the protein polynucleotide barcode, or both are 1-25, 2-20, 3-15, or 4-10 nucleotides in length. Barcodes may form a portion of a longer polynucleotide that includes additional sequences or structural elements. In embodiments, complexes provided herein include a substrate polynucleotide barcode that forms a portion of a polynucleotide that is 10-100, 10-50, or 20-30 nucleotides in length; and/or a protein polynucleotide barcode that forms a portion of a polynucleotide that is 10-100, 10-50, or 20-30 nucleotides in length. In embodiments, a polynucleotide comprising a polynucleotide barcode is 10-50 nucleotides in length. In embodiments, a polynucleotide comprising a polynucleotide barcode is 20-30 nucleotides in length. Examples of additional sequences include, but are not limited to, sequences that participate in the formation of a hairpin structure (e.g., a loop sequence and a self-complementary 3′ end), and a primer binding sequence that is complementary to at least a portion of a primer (e.g., a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length.

In embodiments, generating an identification oligonucleotide further includes amplifying the identification oligonucleotide to generate amplification products. In embodiments, the cleaved complex is amplified via solid phase amplification (e.g., bridge amplification). In embodiments, amplifying includes a plurality of cycles of strand denaturation, primer hybridization, and primer extension. In embodiments, amplifying includes thermally cycling between (i) about 80-95° C. for about 15-30 sec for denaturation, and (ii) about 50-75° C. for about 1 minute for annealing/extension of the primer. In embodiments, amplifying includes thermally cycling between about 72-80° C. for about 5 seconds to about 30 seconds for denaturation; and (ii) about 60-70° C. for about 30 to 90 seconds for annealing/extension of the primer. In embodiments, amplifying includes thermally cycling between (i) about 67-80° C. for about 5 seconds to about 30 seconds for denaturation; and (ii) about 60-70° C. for about 30 to 90 seconds for annealing/extension of the primer. In embodiments, amplifying includes thermally cycling between about 35° C. and about 65° C. In embodiments, amplifying includes thermally cycling between about 40° C. and about 60° C. In embodiments, amplifying includes thermally cycling between about 40° C. and about 58° C. In embodiments, amplifying includes thermally cycling between about 42° C. and about 62° C. In embodiments, amplifying includes thermally cycling between 35° C. and 65° C. In embodiments, amplifying includes thermally cycling between 40° C. and 60° C. In embodiments, amplifying includes thermally cycling between 40° C. and 58° C. In embodiments, amplifying includes thermally cycling between 42° C. and 62° C. In embodiments, amplifying includes thermally cycling about +/−45° C. In embodiments, amplifying includes thermally cycling about +/−40° C. In embodiments, amplifying includes thermally cycling about +/−35° C. In embodiments, amplifying includes thermally cycling about +/−30° C. In embodiments, amplifying includes thermally cycling about +/−25° C. In embodiments, amplifying includes thermally cycling about +/−20° C. In embodiments, amplifying includes thermally cycling about +/−15° C. In embodiments, amplifying includes thermally cycling about +/−10° C. In embodiments, amplifying includes thermally cycling about +/−5° C. In embodiments, amplifying includes thermally cycling about +/−2° C. In embodiments, a device as described herein is configured to perform amplifying of a polynucleotide. In embodiments, amplifying includes RCA or eRCA amplification.

The polynucleotide (i.e., identification oligonucleotide) can be amplified by a suitable method. In embodiments amplifying includes a suitable thermal stable polymerase. Thermal stable polymerases are known in the art and are stable for prolonged periods of time, at temperature greater than 80° C. when compared to common polymerases found in most mammals. Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

In embodiments, amplifying includes a ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction (CCR), and the like.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

In embodiments, amplifying includes RCA or eRCA amplification. RCA refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (eRCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” or “cluster” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments, the second biomolecule-specific binding agent further includes a detectable moiety. In embodiments, the second biomolecule-specific binding moiety further includes a bioconjugate reactive moiety, an enzyme, or a label. In embodiments, the second biomolecule-specific binding moiety further includes a bioconjugate reactive moiety (e.g., a moiety capable of covalently reacting with a second bioconjugate reactive moiety to form a bioconjugate linker). In embodiments, the second biomolecule-specific binding moiety further includes an enzyme. In embodiments, the second biomolecule-specific binding moiety further includes a label (e.g., a fluorescent dye).

In embodiments, the second biomolecule-specific binding agent further comprises a detectable moiety that is a bioconjugate reactive moiety which forms a bioconjugate (e.g., covalent linker) between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate). In embodiments, the association can be direct and formed using bioconjugate chemistry (i.e., the association of two bioconjugate reactive groups) through, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). In embodiments, the first bioconjugate reactive group is a maleimide moiety covalently attached to the second bioconjugate reactive group, a sulfhydryl. In embodiments, the first bioconjugate reactive group is haloacetyl moiety is covalently attached to the second bioconjugate reactive group, asulfhydryl. In embodiments, the first bioconjugate reactive group is a pyridyl moiety covalently attached to the second bioconjugate reactive group, a sulfhydryl. In embodiments, the first bioconjugate reactive group is a —N-hydroxysuccinimide moiety covalently attached to the second bioconjugate reactive group, an amine. In embodiments, the first bioconjugate reactive group is maleimide moiety covalently attached to the second bioconjugate reactive group, a sulfhydryl. In embodiments, the first bioconjugate reactive group is -sulfo-N-hydroxysuccinimide moiety covalently attached to the second bioconjugate reactive group, an amine.

In embodiments, the bioconjugate reactive moiety may include carboxyl groups and various derivatives thereof. In embodiments, the bioconjugate reactive moiety includes N-hydroxysuccinimide esters, N-hydroxybenztriazole esters, acid halides, acyl imidazoles, thioesters, p-nitrophenyl esters, alkyl, alkenyl, alkynyl or aromatic esters. In embodiments, the bioconjugate reactive moiety includes hydroxyl groups which can be converted to ester(s), ether(s), aldehyde(s). In embodiments, the bioconjugate reactive moiety includes haloalkyl group(s), whereby the halide can be later displaced with a nucleophilic group such as, for example, an amine, a carboxylate anion, thiol anion, carbanion, or an alkoxide ion, thereby resulting in the covalent attachment of a new group at the site of the halogen atom. In embodiments, the bioconjugate reactive moiety includes dienophile groups, which are capable of participating in Diels-Alder reactions such as, for example, maleimido or maleimide groups. In embodiments, the bioconjugate reactive moiety includes aldehyde or ketone groups such that subsequent derivatization is possible via formation of carbonyl derivatives such as, for example, imines, hydrazones, semicarbazones or oximes, or via such mechanisms as Grignard addition or alkyllithium addition. In embodiments, the bioconjugate reactive moiety includes sulfonyl halide groups for subsequent reaction with amines, for example, to form sulfonamides. In embodiments, the bioconjugate reactive moiety includes thiol groups, which can be converted to disulfides, reacted with acyl halides, or bonded to metals such as gold, or react with maleimides. In embodiments, the bioconjugate reactive moiety includes amine or sulfhydryl groups (e.g., present in cysteine), which can be, for example, acylated, alkylated or oxidized. In embodiments, the bioconjugate reactive moiety includes alkenes, which can undergo, for example, cycloadditions, acylation, Michael addition, etc. In embodiments, the bioconjugate reactive moiety includes epoxides, which can react with, for example, amines and hydroxyl compounds. In embodiments, the bioconjugate reactive moiety includes phosphoramidites and other standard functional groups useful in nucleic acid synthesis. In embodiments, the bioconjugate reactive moiety includes metal silicon oxide bonding. In embodiments, the bioconjugation reaction includes metal bonding to reactive phosphorus groups (e.g., phosphines) to form, for example, phosphate diester bonds. In embodiments, the bioconjugation reaction includes an azide coupled to an alkyne using copper catalyzed cycloaddition click chemistry. In embodiments, the bioconjugation reaction includes an azide coupled to an alkyne using copper-free click chemistry. In embodiments, the bioconjugate reactive moiety includes biotin conjugate can react with avidin or streptavidin to form an avidin-biotin complex or streptavidin-biotin complex.

In embodiments, the detectable moiety is an enzyme. In embodiments, the enzyme is horseradish peroxidase (HRP) or alkaline phosphatase (AP). In embodiments, the enzyme can be biotinylated. The biotinylated enzymes can bind strongly to avidin and streptavidin proteins. In embodiments, the HRP or AP enzymes are conjugated to either the avidin or streptavidin protein. The HRP or AP enzymes are capable of producing a detectable product (e.g., a luciferase or a peroxidase) from substrates and quantification may include a measurement of the detectable product. In embodiments, enzyme substrates that can be used are chromogenic ELISA substrates, such as 4-chloro-1-naphtho, (4-CN), 3,3′-diaminobenzidine (DAB), 3,3′,5,5′-tetramethylbenzidine (TMB), nitroblue tetrazolium (NBT); chemiluminescent ELISA substrates, such as luminol and disodium 2-chloro-5-(4-methoxyspiro{1,2-dioxetane-3,2′-(5′-chloro)tricyclo[3.3.1.1]decan}-4-yl)-1-phenyl phosphate (CDP-Star); and fluorescent ELISA substrates (e.g., fluorescent dyes, such as Atto dyes).

In embodiments, the second biomolecule-specific binding agent includes a detectable moiety. In embodiments, the second biomolecule-specific binding agent further includes a label also referred to as a dye. In embodiments, the label may be a fluorescent label. Quantification of the label may include a measure of fluorescence intensity, which may be compared to reference values or an internal control. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is luciferin that reacts with luciferase to produce a detectable signal in response to one or more bases being incorporated into an elongated complementary strand, such as in pyrosequencing.

In embodiments, detecting the biomolecule includes detecting the identification oligonucleotide or one or more of the amplification products present in the cleaved complex (or an amplification product including the identification oligonucleotide). In embodiments, detecting the biomolecule includes detecting the detectable moiety of the second biomolecule-specific agent.

In embodiments, detecting the solid support includes detecting the two or more fluorescent dyes of the solid support. In embodiments, detecting the solid support includes detecting the spectrum signature of the solid support.

In embodiments, the solid support includes a substrate polynucleotide barcode, or the first biomolecule-specific binding agent includes a substrate barcode. In embodiments, detecting the solid support includes detecting substrate barcode. In embodiments, detecting includes sequencing. In embodiments, detecting includes hybridizing a labeled probe (e.g., an oligonucleotide including a detectable moiety having a complementary sequence to the substrate barcode). In embodiments, the 5′ end of the substrate barcode contains a functional group that serves to attach the barcode polynucleotide to the substrate via a bioconjugate linker. Non-limiting examples of covalent attachment include amine-modified polynucleotides reacting with epoxy or isothiocyanate groups on the substrate, succinylated polynucleotides reacting with aminophenyl or aminopropyl functional groups on the substrate, dibenzocycloctyne-modified polynucleotides reacting with azide functional groups on the substrate (or vice versa), trans-cyclooctyne-modified polynucleotides reacting with tetrazine or methyl tetrazine groups on the substrate (or vice versa), disulfide modified polynucleotides reacting with mercapto-functional groups on the substrate, amine-functionalized polynucleotides reacting with carboxylic acid groups on the core via 1-ethyl-3-(3-dimethylaminopropyl)-carbodiimide hydrochloride (EDC) chemistry, thiol-modified polynucleotides attaching to a substrate via a disulfide bond or maleimide linkage, alkyne-modified polynucleotides attaching to a substrate via copper-catalyzed click reactions to azide functional groups on the substrate, and acrydite-modified polynucleotides polymerizing with free acrylic acid monomers on the substrate to form polyacrylamide or reacting with thiol groups on the substrate.

In embodiments, the solid support (e.g., the particle) includes one or more immobilization oligonucleotides having complementary sequences to immobilized oligonucleotide attached to the well. The immobilization oligonucleotide may be 10-50 nucleotides, useful for retaining the particle in the well. In embodiments, the immobilization oligonucleotide includes 40-50 nucleotides.

In embodiments, the immobilization oligonucleotide and the substrate barcode are attached to the solid support via a linker (e.g., a non-cleavable covalent linker). The linkers may also include spacer nucleotides. In embodiments, the linker includes 10 spacer nucleotides. In embodiments, the linker includes 12 spacer nucleotides. In embodiments, the linker includes 15 spacer nucleotides. It is preferred to use polyT spacers, although other nucleotides and combinations thereof can be used. In embodiments, the linker includes 10, 11, 12, 13, 14, or 15 T spacer nucleotides. In embodiments, the linker includes 12 T spacer nucleotides. Spacer nucleotides are typically included at the 5′ ends of polynucleotides which are attached to a suitable support. Attachment can be achieved via a phosphorothioate present at the 5′ end of the polynucleotide, an azide moiety, a dibenzocyclooctyne (DBCO) moiety, or any other bioconjugate reactive moiety. The linker may be a carbon-containing chain such as those of formula —(CH₂)ₙ— wherein “n” is from 1 to about 1000. In embodiments, the linker includes polyethylene glycol (PEG) having a general formula of —(CH₂—CH₂—O)ₘ—, wherein m is from about 1 to 500, 1 to 100, or 1 to 12. In embodiments, detecting the solid support includes sequencing the substrate barcode.

In embodiments, the method includes detecting the solid support by sequencing the substrate barcode, and detecting the cleaved complex includes hybridizing a labeled oligonucleotide to the cleaved complex. In embodiments, the method includes detecting the solid support by sequencing the substrate barcode, and detecting the cleaved complex includes hybridizing an oligonucleotide to the cleaved complex and extending the cleaved complex by incorporated one or more labeled nucleotides. In embodiments, the method includes detecting the solid support by sequencing the substrate barcode, and detecting the cleaved complex includes amplifying the cleaved complex and detecting the amplification products (e.g., hybridizing one or more fluorescent probes to the amplification products and detecting the probes). In embodiments, the method includes detecting the solid support by sequencing the substrate barcode; and detecting the cleaved complex includes amplifying the cleaved complex and sequencing the amplification products.

In embodiments, the solid support is a particle including two or more fluorescent dyes within or on the particle. For example, the particle may be a multicolored, fluorescently stained small particles of generally less than 100 μm in diameter (e.g., approximately 10 nm to 100 μm in diameter), wherein the ratio of fluorescent dyes creates a unique spectrum signature for each particle that may be known and thus associated with the capture antibody. The specific ratio or proportion of dyes within a population of particles will determine the location of said populations on a fluorescence map of a multiwell container (e.g., an array), which allocates these populations according to fluorescent color and brightness. By using as little as two dyes, e.g., orange and red, as many as 64 populations of beads are made each one distinct from another by subtle variations in unique fluorescence characteristics recognized by standard detection devices (e.g., flow cytometry instrument). In embodiments, the solid support is a polymeric microsphere (e.g., polystyrene or latex). Any type of polymeric material of microspheres is acceptable including but not limited to brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, or combinations thereof. The particles may also include 1 to 30% of a cross-linking agent, such as divinyl benzene, ethylene glycol dimethacrylate, trimethylol propane trimethacrylate, or N,N′methylene-bis-acrylamide or other functionally equivalent agents known in the art. In embodiments, the particles are made of polystyrene and contain 1 to 30% divinyl benzene. See for example, the particles and methods of making particles as described in U.S. Pat. Nos. 6,632,526 and/or 8,568,881, each of which are incorporated herein in their entireties.

In embodiments, the solid support is a particle including a plurality of dyes (e.g., two or more dyes). In embodiments, the two or more fluorescent dyes are selected from cyclobutenedione derivatives, symmetrical and unsymmetrical squaraines, substituted cephalosporin compounds, fluorinated squaraine compositions, alkylalkoxy squaraines, or squarylium compounds. Squarylium compounds or squaraines as used herein refer to fluorescent compounds derived from squaric acid containing a cyclobutene moiety flanked between two aromatic moieties. In embodiments, the two or more fluorescent dyes are selected from 1,3-bis [(1,3-dihydro-1,3,3-trimethyl-2H-indol-2-ylidene)methyl]-2,4-dihydroxycyclobutenediylium, bis(inner salt) and 2-(3,5-dimethylpyrrol-2-yl)-4-(3,5-dimethyl-2H-pyrrol-2-ylidene)-3-hydroxy-2-cyclobuten-1-one. The molar ratio between first and second dye, when present in a particle, may be between about 0 and 10,000, more preferably between 0.00001 and 2,000. Both dyes would preferably be excited at the same absorption wavelength, e.g., ranging from ultraviolet to about 800 nm, and emit fluorescent light at two spectrally distinct wavelengths, which are spectrally resolved by at least 10 nm, preferably 30 nm, and more preferably by at least 50 nm. For example, the emission peak of the dye #1 is at 585 nm, and the peak emission of dye #2 is at 630 nm. In embodiments, the particle is a fluorescent particle. In embodiments, detecting the solid support includes detecting the particle. In embodiments, detecting the solid support includes detecting the fluorophore on or within the particle. In embodiments, detecting the solid support includes detecting the one or more fluorophores on or within the particle. In embodiments, detecting the solid support includes detecting the spectrum signature of one or more fluorophores of on or within the particle. In embodiments, detecting the solid support includes detecting the emission spectrum of the particle. In embodiments, the method includes detecting the solid support by detecting the fluorescent particle, and detecting the cleaved complex includes amplifying the cleaved complex and detecting the amplification products (e.g., hybridizing one or more fluorescent probes to the amplification products and detecting the probes).

In an aspect is provided a method of amplifying a polynucleotide sequence attached to a biomolecule-specific binding agent, the method including: contacting a solid support with a biomolecule, wherein the solid support includes: a first biomolecule-specific binding agent attached to the solid support; a second biomolecule-specific binding agent attached to the solid support via a cleavable linker, wherein the cleavable linker includes the polynucleotide sequence; forming a complex including a biomolecule bound to both the first biomolecule-specific binding agent bound and the second biomolecule-specific binding agent; cleaving the cleavable linker of the complex thereby forming a cleaved complex; wherein cleaving includes contacting the cleavable linker with a cleaving agent and detaching the second biomolecule-specific binding agent from the solid support; and hybridizing a primer oligonucleotide to the polynucleotide sequence and extending the primer oligonucleotide sequence with a polymerase, thereby amplifying the polynucleotide sequence attached to the second biomolecule-specific binding agent.

In an aspect is provided a method of amplifying a polynucleotide sequence attached to an analyte-specific binding agent, the method includes: contacting a solid support with an analyte, wherein the solid support includes: a first analyte-specific binding agent attached to the solid support; a second analyte-specific binding agent attached to the solid support via a cleavable linker, wherein the cleavable linker includes the polynucleotide sequence; forming a complex includes an analyte bound to both the first analyte-specific binding agent bound and the second specific-binding agent; cleaving the cleavable linker of the complex thereby forming a cleaved complex; wherein cleaving includes contacting the cleavable linker with a cleaving agent and detaching the second analyte-specific binding agent from the solid support; and hybridizing a primer oligonucleotide to the polynucleotide sequence and extending the primer oligonucleotide sequence with a polymerase, thereby amplifying the polynucleotide sequence attached to the second analyte-specific binding agent. In embodiments, the cleavable linker is a covalent linker.

In embodiments, amplifying includes RCA (e.g., extension of a circular polynucleotide with a strand-displacing enzyme). RCA amplifies a circular template and generates long DNA strands that collapse into bundles of DNA. These bundles can be visualized by hybridizing fluorophore-labelled oligonucleotides to quantify the number and intensity of dots by fluorescence microscopy, or by enzyme-labeled detection oligonucleotides, making it possible to detect single molecules.

In embodiments, the primer oligonucleotide is hybridized to the polynucleotide sequence and extended with a polymerase. Primer binding sequences usually have a length in the range of between 3 to 36 nucleotides, also 5 to 24 nucleotides, also from 12 to 36 nucleotides.

In embodiments, the primer oligonucleotide is hybridized to the polynucleotide sequence of the cleaved complex and extended with a polymerase by a padlock probe. Typically padlock probes hybridize to adjacent sequences and are then ligated together to form a circular oligonucleotide. The oligonucleotide primers hybridize to sequences adjacent to the target nucleic acid sequence resulting in a gap (e.g., a gap spanning the length of a nucleic acid sequence, such as a UMI sequence). Padlock probes are specialized ligation probes, examples of which are known in the art, see for example Nilsson M, et al. Science. 1994; 265(5181):2085-2088), and Christian A T, et al. Proc Natl Acad. Sci USA. 2001; 98(25):14238-14243, both of which are incorporated herein by reference in their entireties. In embodiments, the padlock probe (PLP) is a single-stranded oligonucleotide containing a first complementary region and a second complementary region (i.e., nucleic acid sequences complementary to nucleic acid sequences flanking the target nucleic acid sequence of the cleaved complex). In embodiments, the padlock probe further includes an amplification priming site (i.e., a nucleic acid sequence complementary to an amplification primer) and a distinct sequencing priming site (i.e., a nucleic acid sequence complementary to a sequencing primer). Alternatively, in embodiments, the padlock probe further includes an amplification priming site and a sequencing priming site that are the same, are partially overlapping, or in which one is internal to the other. The amplification products are then detected, for example, by hybridizing a sequencing primer to one or more sequencing primer binding sequences on the amplification product and incorporated a labeled nucleotide. Alternative modes of detection are contemplated herein, for example FISH, SBB, and the like. In embodiments, the primer binding sequence is complementary to a fluorescent in situ hybridization (FISH) probe. FISH probes may be custom designed using known techniques in the art, see for example Gelali, E., et al. Nat Commun. 10, 1636 (2019).

In embodiments, the method further includes quantifying biomolecules present by measuring the relative signal intensity of the cleaved complex upon fluorescent detection. In embodiments, detecting the biomolecule includes measuring a level of the biomolecule in the sample. In embodiments, measuring the level of the biomolecule in the sample includes forming a plurality of complexes and quantifying the amount of the label present in the complexes. The method of quantifying the label will depend on the nature of the label selected. A variety of suitable labels are available. For example, where the label is a fluorescent label, quantification may include a measure of fluorescence intensity, which may be compared to reference values or an internal control. As a further example, where the label is an enzyme that produces a detectable product (e.g., a luciferase or a peroxidase), quantification may include a measurement of the detectable product. In another example, the label is a fluorescently-tagged nucleotide, such that measurement of fluorescence intensity and wavelength in a sequencing reaction permits simultaneously determining the sequence of a barcode polynucleotide and the absolute or relative amount of that barcode, and the amount of an associated biomolecule-specific binding moiety or biomolecule.

In embodiments, a biomolecule is deemed to be detected, or is measured, only when one or both of the substrate polynucleotide barcode or the cleaved complex is detected. In embodiments, a biomolecule is deemed to be detected, or is measured, only when both the substrate polynucleotide barcode and the cleaved complex is detected.

In embodiments, the method further includes sequencing the amplification product(s) and/or the substrate barcode. Sequencing includes, for example, detecting a sequence of signals within the particle. Examples of sequencing include, but are not limited to, sequencing by synthesis (SBS) processes in which reversibly terminated nucleotides carrying fluorescent dyes are incorporated into a growing strand complementary to the target strand being sequenced.

In embodiments, the nucleotides are labeled with up to four unique fluorescent dyes. In embodiments, the readout is accomplished by epifluorescence imaging. A variety of sequencing chemistries are available, and non-limiting examples of which are described herein.

In embodiments, the method further includes sequencing the amplification products or complements thereof. In embodiments, sequencing includes a sequencing-by-synthesis or sequencing-by-binding process. In embodiments, sequencing includes hybridizing a sequencing primer to the amplification product, or a complement thereof; contacting the sequencing primer with a sequencing solution including one or more modified nucleotides including a reversible terminator; and monitoring the sequential incorporation of complementary nucleotides to generate one or more sequencing reads, wherein the reversible terminator is removed prior to the introduction of the next complementary nucleotide. In embodiments, sequencing includes hybridizing a sequencing primer to the amplification product, or a complement thereof; incorporating one or more modified nucleotides including a reversible terminator into the sequencing primer with a polymerase to create an extension strand; and detecting the one or more incorporated nucleotides so as to identify each incorporated nucleotide in the extension strand, thereby generating one or more sequencing reads, wherein the reversible terminator is removed prior to the introduction of the next complementary nucleotide.

In embodiments, the sequencing includes sequencing-by-synthesis, sequencing by ligation, or pyrosequencing. In embodiments, generating a first sequencing read or a second sequencing read includes a sequencing by synthesis process. In embodiments, sequentially sequencing the amplification clusters includes generating a plurality of sequencing reads. In embodiments, sequentially sequencing the amplification clusters produces one or more sequencing reads. In embodiments, monitoring the sequential incorporation of complementary nucleotides includes a sequencing-by-synthesis, sequencing-by-ligation, or sequencing-by-binding process. In embodiments, monitoring the sequential incorporation of complementary nucleotides includes incorporating one or more modified nucleotides into the sequencing primer with a polymerase to create an extension strand, and detecting the one or more incorporated nucleotides to identify each incorporated nucleotide in said extension strand, thereby generating one or more sequencing reads.

In embodiments, generating a sequencing read includes executing a plurality of sequencing cycles, where each cycle includes extending the sequencing primer by incorporating a nucleotide or nucleotide analogue using a polymerase and detecting a characteristic signature indicating that the nucleotide or nucleotide analogue has been incorporated.

In embodiments, the method includes sequencing the first and/or the second strand of a double-stranded amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which are incorporated herein by reference in their entireties). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids and amplicons thereof that are present at features of an array are subjected to repeated cycles of oligonucleotide delivery and detection. SBL methods include those described in Shendure et al. Science 309:1728-1732 (2005); U.S. Pat. Nos. 5,599,675; and 5,750,341, each of which are incorporated herein by reference in their entireties. SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which are incorporated herein by reference in their entireties.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes); U.S. Pat. No. 5,688,648 (energy transfer dyes); and the like.

In embodiments, sequencing is performed according to a “sequencing-by-binding” method (see, e.g., U.S. Pat. Pubs. US2017/0022553 and US2019/0048404, each of which are incorporated herein by reference in their entireties), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

### Examples

### Example 1. Multiplexing Assay

Several methods have been developed that can detect very low concentrations of analyte molecules. For example, in sandwich enzyme-linked immunosorbent assay (ELISA), an immobilized antibody is used to capture an analyte followed by binding of a detectable, second antibody (i.e., forming a capture antibody-analyte-detection antibody sandwich). Sandwich immunoassay techniques provide highly sensitive and specificity for target analytes (e.g., antibodies, antigens, proteins, glycoproteins, and hormones) since two antibodies are used for capture (Ab_{C}) and detection (Ab_{D}). The detection antibody, Ab_{D}, can be enzyme conjugated (direct sandwich ELISA) or when the detection antibody used is unlabeled, a secondary enzyme-conjugated detection antibody is required for detection (indirect sandwich ELISA). When performed in a 96-well microtiter plate, typical sandwich immunoassays provide efficient, detection of analytes, such as tacrolimus (Wei T Q, Zheng Y F, Dubowy M, Sharma M. Clin Chem. 2014; 60:621-630), angiotensin II (Towbin H, Motz J, Oroszlan P, Zingel O. J Immunol Methods. 1995; 181:167-176), dextran (Wang S Y, Li Z, Wang X J, Lv S, Yang Y, Zeng L Q, Luo F H, Yan J H, Liang D F. Monoclon Antib Immunodiagn Immunother. 2014 October; 33(5):334-9), and naringin (Qu H, Wang X, Qu B, Kong H, Zhang Y, Shan W, Cheng J, Wang Q, Zhao Y. Anal Chim Acta. 2016 Jan. 15; 903:149-55). The ability to multiplex, that is, simultaneously detect different analytes remains a significant challenge. Simultaneous measurement of multiple analytes from a single sample will result in a significant cost, time, and sample savings. Optimizing the detection antibody concentration and minimizing non-specific binding and cross-reactivity are useful to generate a multiplexed immunoassay.

Early disease diagnosis plays an important role in effective treatment. Clinical evidence based on a single analyte or single biomarker is typically not adequate for a confident diagnosis of a disease or monitoring treatment. In a typical multiplexed sandwich immunoassay, a false signal can be produced when an incorrect detection antibody binds to a bead or an array element that is coated with a capture antibody. While commercial single-plex techniques, such as enzyme-linked immunosorbent assay (ELISA) (e.g., sandwich immunoassays) and biomarker kits, can accurately detect a single analyte, the monitoring of more complex, multifactorial diseases, such as cancer, autoimmune, and neurodegenerative diseases, require the analysis of multiple biomarkers in order to confidently address the underlying disease (e.g., deciding best treatment options, tracking disease progression and response to therapy, and formulating prognoses). Multiplex immunoassays confer several advantages over widely used single-plex assays including increased efficiency, greater output per sample volume, and higher throughput.

Commercial multiplex immunoassays include arraying different capture antibodies at different locations on a planar surface (e.g., a glass slide) or within wells of a microtiter plate. Typically, each feature (i.e., a spot on the slide or well on the microtiter plate) includes a mixture of different Ab_{C} immobilized to the surface. Alternatively, multiplex bead suspension assays have been developed, wherein a plurality of different Ab_{C} are immobilized to the surface of a fluorescently activated plastic bead and detected via flow cytometric methods, for example fluorescence-activated cell sorting (FACS) methods; see for example Martins, T. B., Burlingame, R., von Mühlen, C. A., Jaskowski, T. D., Litwin, C. M., & Hill, H. R. (2004). Clinical and diagnostic laboratory immunology, 11(6), 1054-1059; and Kellar K L, Mahmutovic A J, Bandyopadhyay K. Curr Protoc Cytom. 2006 February; Chapter 13: Unit13.1). In the multiplex bead system, capture antibodies that specifically bind to target analytes are immobilized to uniquely labeled (e.g., barcoded or layer-by-layer (multilayer) fluorescence-encoded) beads. By decoding the beads and detecting the captured analyte using a Ab_{D}, the identity of captured analytes can be determined.

Despite commercial successes, a major challenge is cross-reactivity (i.e., non-specificity) of detection antibodies binding to incorrect analytes, which results in false positives, false negatives, and/or an increase in background noise. The vulnerability of multiplex sandwich assays to cross-reactivity increases quadratically with the number of targets (Pla-Roca M, et al. Mol Cell Proteomics. 2012 April; 11(4):M111.011460). Though theoretically thousands of different targets may be detected, researchers determined the practical limit for multiplexing to 30-50 targets of purified (i.e., non-interfering biomarkers removed) sample (Perlee, L. T., et al. (2004) Proteome science, 2, 9). To date, detection of 30-50 targets represents the upper limit (Gunther, A., et al. (2020) Frontiers in Immunology, 11, 572634). While carefully selected antibodies have a high degree of specificity, they are not infinitely specific. Most antibodies will non-specifically bind different proteins with low affinity, which limits the degree of multiplexing that can be achieved while avoiding non-specific interactions. This is one reason why most currently available commercial multiplexed assay panels are limited to 10-20 analytes or fewer. Additionally, developing two antibodies possessing different epitopes for each target analyte is time-consuming, challenging, and expensive. A significant amount of expensive reagent (i.e., Ab_{D}) is wasted if the target analyte is not captured from the sample.

To address the need for improved multiplex protein detection methods, provided herein are immunoassays where the identity of both the capture and detection antibody are colocalized (i.e., located in the same physical location) and detected. This enables a much higher degree of specificity than previously achievable when measuring analytes simultaneously. The approach also offers an almost unlimited degree of multiplexing through the readout of polynucleotide barcodes. For example, described herein are compositions, substrates, and methods to address these problems. For example, described herein is a substrate that includes a plurality of specific capture agents and a plurality of specific detection agents. The specific detection agents are immobilized to the substrate via a removable tether. In embodiments, the tether includes a polynucleotide sequence, which may be amplified and detected.

A general workflow for preparing the assay is as follows: a multiwell container was provided and a plurality of immobilization oligonucleotides are bound to the wells (e.g., 0.1 μM of immobilization oligonucleotides containing a DBCO moiety are reacted with an azide containing polymer coated well and incubated for 30 minutes at room temperature). A plurality of particles was provided to the multiwell container (e.g., 0.0025 mg/mL of particles per well). The particles include a capture antibody immobilized to the particle via a PEG linker (e.g., PEG3 or PEG12 linker) and a detection antibody immobilized to the particle via a cleavable linker. The particle also contained a substrate barcode. The cleavable linker may include multiple cleavable sites to ensure cleavage and maximize the signal-to-noise ratio. For example, the cleavable linker may have the following sequence: 5′-AAAAAAAAATCGTTCTACTACGCG GTCTCGTTCGACCGTGCGGTTUUUAAAAAAAUUUAAAAAAAUUUAAAAAAA-3′ (CL-1, SEQ ID NO:1), wherein the trio of uracils (i.e., UUU) represent the cleavable sites cleavable via enzymatic (e.g., Uracil-DNA glycosylase (UDG) and Endonuclease VIII (EndoVIII)) and/or chemical cleaving agents. Alternative cleavable tethers utilized herein are provided in Table 1, all of which are efficiently cleaved using an enzymatic cocktail.

TABLE 1
Cleavable linkers useful for attaching
the detection antibody to
the particle.

| | |
|---|---|
| CL-2 | 5′-AAAAAAAAATCGTTCTACTACGCGGT |
| (SEQ ID NO: 2) | CTCGTTCGACCGTGCGGTTUAAAAAAA-3′ |
| CL-3 | 5′-AAAAAAAAATCGTTCTACTACGCGGT |
| (SEQ ID NO: 3) | CTCGTTCGACCGTGCGGTTUUUAAAAAA |
| | A-3′ |
| CL-4 | 5′-AAAAAAAAATCGTTCTACTACGCGG |
| (SEQ ID NO: 4) | TCTCGTTCGACCGTGCGGTTUUUUUAAA |
| | AAAA-3′ |
| CL-5 | 5′-AAAAAAAAATCGTTCTACTACGCGG |
| (SEQ ID NO: 5) | TCTCGTTCGACCGTGCGGTTUUUUU-3′ |
| CL-6 | 5′-AAA AAA AAA TCG TTC TAC |
| (SEQ ID NO: 6) | TAC GCG GTC TCG TTC GAC |
| | CGT GCG GTT UUU AAAAAAA |
| | UUU AAAAAAA-3′ |
| CL-7 | 5′-AAA AAA AAA TCG TTC TAC |
| (SEQ ID NO: 7) | TAC GCG GTC TCG TTC GAC CGT |
| | GCG GTT UUU AAA AAA AAA AAA |
| | AA UUU AAAAAAA-3′ |

The particle-loaded array was imaged to confirm particle loading, and subsequently incubated with a blocking buffer, e.g., 5% Bovine serum albumin (BSA), to facilitate target protein binding while minimizing non-specific binding. Samples containing different concentrations of the analyte, IL-6, were incubated (e.g., 10, 1, 0.5, 0.25, 0.1, 0 ng/mL) with the particle-loaded array for 30 min at RT, followed by incubation with a cleaving agent to cleave the cleavable linker. A crosslinking agent was added (40 μL of 4% glutaraldehyde) for 10 minutes at RT, followed by detection. The remnant of the cleavable linker (i.e., the cleaved complex) was sequenced in addition to sequencing the substrate barcode to detect the analyte (IL-6) and solid support, respectively.

### Example 2. Simultaneous Detection of Different Analytes Via Antibody Detection

For almost 50 years, immunoassays have allowed for sensitive and specific detection of analytes of interest in biological samples. Detection of antibodies in sera has broad applications for detection and monitoring of infectious diseases, autoimmunity and cancer. Traditional enzyme-linked immunosorbent assays (ELISA) assays detect and measure a single analyte per plate, which presents limitations in sampling volume, cost and labor. The development of highly specific monoclonal antibodies and chemiluminescence detection resulted in ELISA assays that have high ease of use, flexibility and low cost. The growth in proteomics and genomic analysis are driving the need to discover and monitor large numbers of biomarkers indicative of human disease states. Patient monitoring of more complex, multifactorial diseases such as cancers, graft-versus-host disease, autoimmune and neurodegenerative diseases require analysis of multiple biomarkers for optimized therapeutic regimens. Validation of novel biomarkers into multiplex immunoassay panels allows simultaneous measurement of multiple analytes in a single patient sample, thereby minimizing assay costs, time and sample volume while enabling progression monitoring and outcome prediction to anticipate potentially adverse drug reactions.

The methods typically used for multiplex immunoassays fit loosely into two categories: i) capture antibody immobilization on a solid surface in which assays for each analyte are spatially separated and ii) capture antibody immobilization on beads in which assays for each analyte are on a different bead.

Assays commercialized by Luminex®, for example, provide different capture antibodies on sets of beads. Each bead is infused with a single or several chemiluminescent/fluorescent dyes to create a unique fluorescent signature. Numerous sets of beads are prepared, each bead having a separate capture antibody according to the cognate analyte and a unique fluorescent signature enabling identification. The sample and the requisite bead sets are combined and allowed to incubate. Sets of detection antibodies, all of which are individually labelled with a single chemiluminescent or fluorescent reporter, are added to the sample upon completion of incubation and washing stages. Each bead thus accommodates a ‘sandwich’ consisting of the captured target analyte and the cognate reporter-conjugated detection antibody. The bead analyte reporter constructs are then analyzed, for example, in a flow chamber whereby lasers excite the chemiluminescent/fluorescent reporters and emitted light is collected by a series of detectors for quantitative analysis, providing information about the analyte(s) present in the sample.

One approach for multiplexing ELISA sandwich assays is to create an array of different capture antibodies immobilized to a solid support and, following incubation with a sample containing the analytes of interest, contacting the array with detection antibodies. The entire array can be read at once and the location of the capture antibodies can be used as an address to determine the analyte being detected. The practicality of such an approach is limited, however, due to cross-reactivity (i.e., non-specificity) of detection antibodies binding to incorrect analytes or capture antibodies, which results in false positives, false negatives, and/or an increase in background noise.

Reported herein is a system in which both the capture antibody (Ab_{C}) and corresponding target-specific detection antibody (Ab_{D}) are colocalized (e.g., simultaneously bound to the same solid support and/or present in the same physical location). For example, in embodiments, the detection antibody is conjugated to the solid support via a cleavable tether that includes a cleavable site. The cleavable tether can optionally include an oligonucleotide sequence. The target analyte will have at least two different non-overlapping antigenic epitopes capable of binding to both the capture antibody and detection antibody. The solid support can be a capture antibody-immobilized bead with a corresponding target-specific detection antibody bound to the same bead. Alternatively, the solid support is a well in a multiwell container. Alternatively, the solid support is a particle in a well in a multiwell container.

In embodiments, to make a detection particle, the solid support includes a first bioconjugate reactive moiety that reacts with a second bioconjugate reactive moiety found on the end of the cleavable tether of the detection antibody to form a bioconjugate linker that serves to covalently attach the detection antibody to the surface of the solid support. The cleavable tether may optionally include one of more oligonucleotide sequences, including, for example, a barcode, primer binding sequence, and/or unique molecular identifier (UMI). Afterwards, the corresponding target-specific capture antibody including a first bioconjugate reactive moiety is bound to the solid support through a second bioconjugate reactive moiety, wherein the second bioconjugate reactive moiety is different from that used to bind the cleavable tether. These solid supports are then washed with buffer solution and optionally blocked (e.g., blocked with BSA) to provide supports that include both immobilized capture antibody and tethered detection antibody. The solid support may be a particle, wherein the particle includes a polymer coating.

In embodiments, the particles are incubated with a sample, for example, serum or plasma which contains the analytes of interest in a buffer solution overnight (16-18 hours) at 4° C. under agitation. During this time, both the analyte-specific capture and detection antibodies recognize and bind to the appropriate epitopes of the corresponding analytes to form a complex. Following overnight incubation, the particles are washed to remove any non-specifically bound entities. The cleavable site included in the Ab_{D} cleavable tether is then exposed to the appropriate cleaving agent. The cleavable site can include a deoxyuracil (dU), for example, that is then cleaved with USER® enzyme mixture. Any suitable enzymatic, chemical, or photochemical cleavage reaction may be used to cleave the cleavable site. This cleavage step is followed by a wash to remove any excess unbound Ab_{D}, leaving behind the corresponding “sandwich” consisting of the immobilized Ab_{C}, captured target analyte, and Ab_{D}.

The analyte of interest can be detected directly by labeling the primary Ab_{D} (e.g., wherein the detection antibody includes a fluorophore) or indirectly by utilizing a labeled secondary antibody. In the direct detection method, the Ab_{D} is labeled with a reporter enzyme, such as horseradish peroxidase (HRP) & alkaline phosphatase (AP) or alternative signaling molecule such as a fluorophore. The indirect detection method involves an additional probing step using another antibody or streptavidin labeled with a detectable tag, such as biotin. The additional probe known as a secondary antibody has the sole purpose of delivering the measurable signal by binding to the primary Ab_{D} for amplification. It is important that the secondary antibody is specific for the Ab_{D} only and not the Ab_{C}. Generally direct detection is less sensitive and has minimal signal amplification relative to indirect detection since each primary Ab_{D} has several epitopes that can be bound by the labeled secondary antibody, allowing for greater signal amplification.

For direct detection step, the “sandwich” complex including the immobilized Ab_{C}, captured target analyte, and Ab_{D} attached to the particle is incubated with a reporter enzyme and an enzyme substrate is introduced to this mixture. The reporter enzyme converts the substrate to a detectable product where the intensity of signal produced will be directly proportional to the amount of antigen captured in the plate and bound by the detection reagents. The choice of substrate depends on instrumentation available for signal-detection. Examples of enzyme substrates that can be used include chromogenic ELISA substrates, such as 4-chloro-1-naphtho, (4-CN), 3,3′-diaminobenzidine (DAB), 3,3′,5,5′-tetramethylbenzidine (TMB), nitroblue tetrazolium (NBT); chemiluminescent ELISA substrates, such as luminol and disodium 2-chloro-5-(4-methoxyspiro{1,2-dioxetane-3,2′-(5′-chloro)tricyclo[3.3.1.1]decan}-4-yl)-1-phenyl phosphate (CDP-Star); and fluorescent ELISA substrates such as traditional fluorescent dyes.

For indirect detection, a biotinylated secondary detection antibody that binds to the Ab_{D} is probed using avidin or streptavidin protein conjugated to either HRP or AP enzymes. Multiple biotin tags per antibody molecule allow for more than one streptavidin molecule to bind so that the number of enzymes in the final immune complex is amplified. Therefore, catalysis of appropriate substrate is increased and will give a stronger signal compared to a conventional enzyme-labeled secondary antibody. The “sandwich” consisting of the immobilized Ab_{C}, captured target analyte and Ab_{D} attached to the particle and biotinylated detection antibody are added together and incubated at room temperature for an hour. The enzyme conjugate is then added and incubated, for example, for another hour before a substrate solution is then added and developed at room temperature for 30 min before absorbance of signal produced is measured using the appropriate detection hardware.

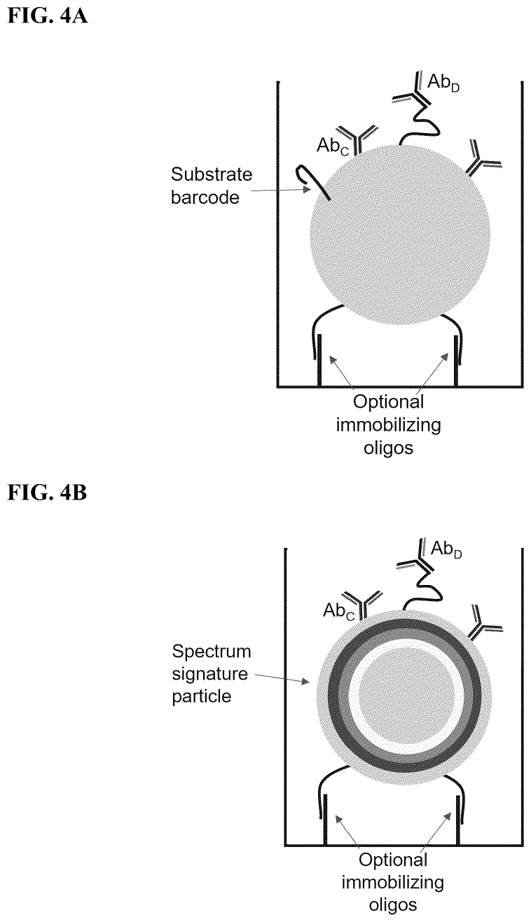

In addition to detecting the detection antibody, the capture antibody may further be detected. For example, the solid support may further include a DNA barcode (e.g., an identifying oligonucleotide sequence associated with the capture antibody), referred to herein as a substrate barcode, as illustrated in FIG. 4A. The DNA barcode may be sequenced or detected via hybridization of a labeled probe. In embodiments, the method includes two sequencing reactions after forming the complex. First, a primer is introduced that allows all the detection barcodes to be read out by sequencing (e.g., sequencing by synthesis). After the barcodes on the detection antibodies are read, the extension can be permanently terminated, or the primers can be removed, or the barcode sequence can be chosen so that the extension is physically limited (i.e. reaches the end of the template). Then, a different primer can be introduced to read out the barcodes on the particle. These barcodes can either be attached directly to the particle, or attached to the capture antibodies. Since all that is required is reading out the identity of the bead, and there is only a single barcode sequence per bead (in multiple copies), this readout can be very short—e.g., five cycles to read out up to 1024 unique beads.

With four possible bases at each position, only a modest number of sequencing cycles is required. For example, in just five bases, 4⁵=1024 combinations can be encoded. Ten bases allow for 4¹⁰, or ˜1 million unique barcodes. Barcodes can be chosen to be as different as possible in the large space of available combinations. In each sequencing cycle, the signal in four (4) fluorescent channels is read out, corresponding to each of the four bases. Once the desired number of sequencing cycles are complete, the linear decomposition of the signal into the known barcode sequences will produce the amplitude (or amount) of each barcoded antibody that was present. Thus, one can discriminate between the signals generated by the specific detection antibodies vs. the non-specific antibodies.

An alternative means for detecting the capture antibody includes detecting the solid support, wherein the solid support is a particle including single or several chemiluminescent/fluorescent dyes to create a unique fluorescent signature (e.g., a Luminex® particle) as illustrated in FIG. 4B. Each particle includes capture antibodies associated with a unique fluorescent signature, thus enabling identification of the substrate and thus the capture antibody.

Examples of assays in which this system could be used include examination of white adipose tissue which is involved in a variety of metabolic and physiological processes. Adipocytes found in this tissue secrete a number of hormones called adipokines with functions that include appetite and energy balance, insulin sensitivity and lipid metabolism. A sample assay kit would include a broad selection of analytes to focus on the therapeutic potential of adipokines. Such a kit can be used for simultaneous quantification of any or all of the following analytes in human plasma or serum samples: HGF, IL-1(3, IL-6, IL-8, insulin, leptin, MCP-1, NGF and TNFα to provide information about the state of the white adipose tissue.

Methods as described herein may be carried out in a number of different formats including beads in a well plate. FIG. 3 shows a set of particles at the bottom of a well plate. Commonly used well plates have 96 or 384 wells. Other well-plate formats, such as 24, 48, 1536, and 9600 wells, etc. could also be used. Each well contains a different antibody system that would bind to a different analyte, thereby allowing capture of variety of analytes as illustrated in FIG. 3. A standard 96-well plate would have a well diameter of approximately 6 mm. If 1-micron diameter particles are used as the capture particles, the surface could accommodate approximately 30 million particles in a monolayer. It may be preferable to use fewer particles to distinctly image individual particles, and thus would be sparsely arranged on the surface. Another approach is to pattern the bottom surface of the well to form a regular array of particles. This would allow a regularly spaced, high-density arrangement of particles, thus maximizing the number of particles that can be distinctly imaged at the desired inter-particle spacing.

Even with larger beads and smaller wells, it would be possible to measure a large number of analytes simultaneously. For example, in a 3 mm diameter well (approximate size for 384-well plates), with 3 μm diameter beads, there would be room for approximately 1 million beads; even a sparse spacing of only 10% coverage would have room for 100,000 beads. With 100 beads representing each analyte, this would allow for 1,000 analytes to be measured simultaneously. In embodiments, about or at least about 100, 500, 1000, 2500, 5000, or more analytes are assayed simultaneously. In embodiments, 100-5000, 250-2500, or 500-1000 analytes are assayed simultaneously. Assaying may include determining the presence, absence, or amount (absolute or relative) of an analyte in a sample.

Such a system can be used with a variety of assays to allow for simultaneous detection of multiple analytes in a single reaction vessel. From a single sample, analytes such as antigens, antibodies, oligonucleotides, enzyme substrates or receptors could all be detected. Detection of broad range of biologically relevant targets in both diseased and healthy control serum, plasma, and tissue culture supernatant samples would allow for proteomic and phenotypic changes associated with a diverse range of pathophysiological conditions.

### Example 3. Simultaneous Detection of Different Analytes Via Sequencing

Currently, profiling proteins in single cells at high throughput requires sensitive and fast methodology. As described above, flow cytometry with fluorescently-labeled antibodies can sensitively profile proteins in millions of single cells. Further, labeling antibodies with dyes of different colors allows profiling to be multiplexed to tens of proteins. However, by swapping out the usual fluorophore or heavy metal-tagged antibodies with DNA sequence tags that could be read at the single-cell level using droplet microfluidic barcoding and DNA sequencing would be advantageous for numerous reasons. Utilization of oligo tags (e.g., barcodes) to label antibodies allows for the amplification and subsequent detection (e.g., detected via sequencing the tags). The oligo tags may include unique molecular identifiers (UMIs) to correct for amplification bias and provide quantitative results. Having the tag identity encoded by its nucleobase sequence provides far more combinations of unique tags than is possible with fluorescence or mass tags. For example, in embodiments when using 4-color detection (i.e., one color per nucleotides) a set of 10 sequencing cycles provides information in 40 dimensions (4 channels per cycle×10 cycles). Any of the up to 4¹⁰ possible barcodes would point to a unique position in this 40-dimensional space. Linear combinations of barcodes are thus easily resolvable, limited only by the accuracy of the sequencing signals. A typical example might be a set of 1,000-10,000 RNA targets, each encoded by a barcode selected from 4N combinations, where N is the number of sequencing cycles or “digits” in the barcode. With 10 cycles, up to 410 or approximately one million barcodes are available. This allows for the ability to select barcodes that are as far apart as possible in the available space (maximizing the Hamming distance), for more robust demultiplexing. The limit to multiplexing would no longer be the availability of unique tag sequences but rather the specific antibodies that could detect the epitopes of interest in a multiplexed reaction. The specific detection antibodies would bind analytes with target epitopes and have a unique sequence tag label. Therefore, the unique oligo tag would be carried with the antibody so the presence of the target can be inferred based on the presence of the tag.

Tuning the conjugation chemistry and increasing the number of DNA tags per antibody can improve the sensitivity of detecting the appropriate analyte. Multiplexing is also important because the proteomes of most organisms are large, including multiple spliceforms and post-translational modifications of just a single gene transcript. Sequence tags allow, theoretically, unique labeling of antibodies against every member of the proteome. This, in turn, would change how experiments are performed, allowing complete and unbiased characterization of the proteome analogous to genome and transcriptome sequencing, and obviating the need to specify which biomarkers to target a priori. Instead, relevant biomarkers would be learned from the data, maximizing the chances of unexpected discoveries.

This method can be applied to surface proteins and also extended to internally-expressed markers since antibodies can bind intracellular targets after permeabilization. The microfluidic method for lysing and barcoding the cells utilizes proteases that digest and solubilize even fixed cells and tissues, freeing the antibody tags for barcoding. This should enable new opportunities for directly detecting the phosphorylation state of proteins in pathways of interest without having to know how they correlate with cell surface expression. Profiling of cell state, by detecting post-translational modification of proteins in relevant cell pathways would also be a possibility.

Specific antibodies tagged with known oligonucleotide sequences can be synthesized by using bifunctional crosslinkers reactive towards thiol (via maleimide) and amine (via NHS) moieties. For example, a 5′-thiol-modified oligonucleotide could be conjugated to a crosslinker via maleimide chemistry and purified. The oligos with a 5′-NHS-ester would then be added to a solution of antibodies and reacted with amine residues on the antibodies surface to generate tagged antibodies capable of binding analytes with target epitopes. These tagged antibodies, hereafter referred to as detection antibodies (Ab_{D}), have a cleavable tether that extends from the oligonucleotide sequence(s). The one or more oligonucleotide sequences may include a barcode, primer binding sequence and/or unique molecular identifier (UMI).

In embodiments, the cleavable tether of the tagged Ab_{D} has a first bioconjugate reactive moiety that is reacted with a particle including a second bioconjugate reactive moiety to covalently attach the tagged Ab_{D} to the surface of the particle. The corresponding target-specific capture antibody (Ab_{C}) comprising a bioconjugate reactive moiety is bound to these particles through another bioconjugate reactive moiety different from that used to bind the cleavable tether of the Ab_{D}. These particles are then washed with buffer solution and optionally blocked (e.g., blocked with BSA) to provide particles comprising both immobilized capture antibody and detection antibody attached through a cleavable tether that comprises one or more oligonucleotide sequences that may include a barcode, primer binding sequence and/or unique molecular identifier (UMI).

Following the same procedure described in Example 2, these particles are incubated with a sample, such as serum or plasma which contains the analytes of interest in buffer solution overnight (16-18 hours) at 4° C. under agitation. During this time, both the analyte-specific capture and detector antibodies recognize and bind to the appropriate epitopes of the corresponding analytes. After overnight incubation, the particles are washed to remove any non-specifically bound analytes. The cleavable site of the cleavable tether on the Ab_{D} is exposed to the appropriate cleaving agent. The cleavable site can have a deoxyuracil (dU), for example, that is then cleaved with USER enzyme mix. Any suitable enzymatic, chemical, or photochemical cleavage reaction may be used to cleave the cleavable site. This cleavage step is followed by a wash to remove any excess unbound Ab_{D} leaving behind the corresponding “sandwich” complex including the immobilized Ab_{C}, captured target analyte, and tagged oligonucleotide Ab_{D} attached to the particle.

FIGS. 2A-2E illustrate means for detecting the complex and thus detecting the analyte. FIG. 2A provides an embodiment wherein following cleavage of the cleavable tether containing a polynucleotide sequence, an extendable 3′ end is formed. This approach provides that the cleavage of the oligonucleotide sequence tethered to the Ab_{D} is required for subsequent signal generation. Thus, any residual un-cleaved tethers containing oligonucleotide sequences will not contribute a background signal. Subsequent detection of the Ab_{D} may then occur following hybridization and detection of a labeled linear oligonucleotide (FIG. 2B), wherein the linear oligonucleotide is already attached to a fluorophore and is detected, or one or more labeled nucleotides are incorporated by extending that 3′ end of the remnant of the tether with labeled nucleotides (i.e., sequencing). Alternatively, a circularizable oligonucleotide may hybridize to the remnant, optional amplifying the circularizable oligonucleotide via rolling circle amplification (e.g., RCA or eRCA), followed by subsequent detection of the circularizable oligonucleotide (FIG. 2C). For example, as depicted in FIG. 2D, the cleaved portion of the tether oligonucleotide sequence serves as the primer for RCA. The circularizable oligonucleotide could either use a fully formed circle, or oligonucleotide capable of being ligated together to form a circle using the tether oligonucleotide sequence both as a splint and a primer. In embodiments, the circularizable oligonucleotide includes a padlock probe (PLP). In embodiments, generating an amplicon with multiple copies of the PLP greatly increases the limit of detection. As illustrated in FIG. 2E, the tether may include a unique molecular identifying (UMI) sequence, wherein the oligonucleotide probe anneals on the UMI, or on flanking positions to the UMI sequence. A polymerase (not shown) extends the end of the oligonucleotide probe thereby incorporating a complement of the UMI into the oligonucleotide probe. The oligonucleotide probe may then be optionally amplified and detected.

For example, one such detection means includes contacting the particle with a circularizable probe, such as a padlock probe (PLP) specific to the oligonucleotide sequence on the Ab_{D} tether remnant (i.e., cleaved complex). The padlock probe consists of linear ssDNA designed to have sequences complementary to two sequences on the target oligonucleotide (e.g., the remnant of the tether, following cleavage). Once the padlock probes bind, the excess is washed away. The 5′ and 3′ ends of the linear strand of the DNA (the PLP) are ligated together to form a circle using the oligonucleotide sequence bound to the Ab_{D} as a “splint”. This only occurs when the two ends of the padlock probe are adjacent to each other.

Alternatively, an oligonucleotide probe including two complementary sequences to the target oligonucleotide (e.g., the remnant of the tether, following cleavage) may be used. The oligonucleotide probe is similar to a padlock probe, however with an important distinction. Typically, padlock probes hybridize to adjacent sequences and are then ligated together to form a circular oligonucleotide. The oligonucleotide probes hybridize to sequences adjacent to the target nucleic acid sequence resulting in a gap (e.g., a gap spanning the length of the target nucleic acid sequence) as observed in FIG. 2E. Padlock probes are specialized ligation probes, examples of which are known in the art, see for example Nilsson M, et al. Science. 1994; 265(5181):2085-2088), and has been applied to detect transcribed RNA in cells, see for example Christian A T, et al. Proc Natl Acad Sci USA. 2001; 98(25):14238-14243, both of which are incorporated herein by reference in their entireties.

The circularizable probe and resulting circle may contain a barcode for reading the identity of the probe and its target. In embodiments, the circularizable probe includes a complementary sequence for binding a sequencing primer, including a site for Rolling Circle Amplification (RCA) priming. The priming site of RCA can have the same sequence as the sequencing primer or some degree of overlap. Optionally the circle can contain multiple repeated barcodes and priming sites. Once the PLP is ligated to form a circle, RCA can amplify a circular polynucleotide by polymerase extension of an amplification primer complementary to a portion of the template polynucleotide. This generates copies of the circular polynucleotide template so that multiple complements of the template sequence are arranged end to end in tandem (i.e. concatemer) locally preserved at the site of the circle formation.

Amplification may occur at isothermal conditions (e.g., RCA or eRCA), or include hybridization chain reaction (HCR) which uses a pair of complementary, kinetically trapped hairpin oligomers to propagate a chain reaction of hybridization events. In embodiments, the amplifying includes branched rolling circle amplification (BRCA), hyberbranched rolling circle amplification (HRCA), which uses a second primer complementary to the first amplification product. This allows products to be replicated by a strand-displacement mechanism, which yields drastic amplification within an isothermal reaction. In embodiments, amplifying includes polymerase extension of an amplification primer. The resulting generated amplicon has multiple copies of the oligonucleotide sequence.

Primers comprising a detectable agent specific to the oligonucleotide sequence can then be added to the particles and hybridize to the target oligonucleotide sequence on the amplicon. Examples of a detectable agent can include imaging agents such as a fluorescent probe, a dye or a reporter enzyme and corresponding substrate whereby the enzyme will convert the substrate into a detectable product. Upon binding of these primers, another wash is performed to remove any unbound primers. Measurement of the detectable agent shows that the appropriate analytes are bound to the particles. Multiple copies of the oligonucleotide sequence allow for highly sensitivity in analyte detection since there will be an increase in signal to noise ratio. Alternatively, primers specific to the oligonucleotide sequence on the Ab_{D} can be added to the particles to allow for sequencing to occur. The resulting sequencing data can be examined to see which barcodes/UMIs are present to provide information about the presence of the corresponding analytes.

### Example 4. Simultaneous Detection of Different Analytes to Evaluate Treatment Response in HER2+ Breast Cancer

HER2+ breast cancer is one of the main subtypes of breast cancers and makes up to 20% of breast cancer cases in women in the United States (www.cancer.org/cancer/types/breast-cancer/understanding-a-breast-cancer-diagnosis/breast-cancer-her2-status.html). In 2019, the FDA approved fam-trastuzumab deruxtecan-nxki (commercially known as Enhertu®) as treatment for patients with unresectable or metastatic HER2+ breast cancer who previously received two or more frontline regimens (See FDA News Release, www.fda.gov/news-events/press-announcements/fda-approves-new-treatment-option-patients-her2-positive-breast-cancer-who-have-progressed-available). Despite these advances, there is a continual need to gain insight into how the HER2+ breast cancer cells evolve in response to Enhertu® as the heterogenous composition of HER2+ breast cancer cells govern how the collective tumor responds to the treatment.

Liquid biopsies is a standard, noninvasive method to monitor disease progression and treatment response from the evaluation of circulating tumor DNA, tumor extracellular vesicles, and circulating tumor proteins (see Wu et al. Cancers (Basel). 2022 Apr. 19; 14(9):2052 and Lone et al. Molecular Cancer. 2022 Mar. 18; 21(79)). The methods described herein could be utilized to provide comprehensive insight regarding treatment response to reveal the relationship between treatment response and, for example, the population(s) of cancer cells undergoing epithelial-mesenchymal transition (EMT) or mesenchymal-epithelial transition (MET), dynamics of cytokine production, and immune biomarkers. Sets of biomolecule-specific capture and detection antibodies could be used with appropriate substrate barcodes or identification oligonucleotides, respectively, to detect up to 12 target proteins germane to HER2+ breast cancer (e.g., epithelial cell surface markers (e.g., CDH1, KRT8, and EpCAM), mesenchymal cell surface markers (e.g., FN, VIM, MMP9, and CTNNB1), inflammatory markers (e.g., IL-6, IL-8, TNF-α, and IFN-γ) and proliferative marker, Ki67). Collective analysis of proteins related to distinct processes to HER2+ breast cancer could provide mechanistic insight about treatment response.

Following the same procedure described supra, samples derived from liquid biopsies are incubated with particles described herein in buffer solution overnight (16-18 hours) at 4° C. under agitation. During this time, both the analyte-specific capture and detection antibodies recognize and bind to the appropriate epitopes of the 12 aforementioned targets. After overnight incubation, the particles are washed to remove any non-specifically bound analytes. The cleavable site of the cleavable tether on the Ab_{D} is exposed to the appropriate cleaving agent. The cleavable site can have a deoxyuracil (dU), for example, that is then cleaved with USER enzyme mix. Any suitable enzymatic, chemical, or photochemical cleavage reaction may be used to cleave the cleavable site. This cleavage step is followed by a wash to remove any excess unbound Ab_{D} leaving behind the corresponding “sandwich” complex including the immobilized Ab_{C}, captured target analyte(s), and tagged oligonucleotide Ab_{D} attached to the particle. Target proteins could be detected by measuring the signal emitted from detectable label present on the analyte-specific detection antibody from each “sandwich” complex. Alternatively, target proteins could be detected by sequencing the identification oligonucleotide (or amplicons thereof) present on the remnant of the cleavable tether from the analyte-specific detection antibody. In addition, the solid support could be detected by reading the substrate barcode present on or in the solid support or measuring the spectrum signature associated with the solid support.

What is claimed is:

1. A method of detecting a biomolecule in a sample, said method comprising:
contacting a solid support with a sample comprising a biomolecule, wherein said biomolecule is a lipid, carbohydrate, peptide, protein, or antigen binding fragment, and said solid support comprises a first biomolecule-specific binding agent attached to the solid support; and a second biomolecule-specific binding agent attached to said solid support via a cleavable linker, thereby forming a complex comprising the first biomolecule-specific binding agent bound to the biomolecule and the second biomolecule-specific binding agent bound to the biomolecule;
cleaving the cleavable linker of the complex thereby forming a cleaved complex;
wherein cleaving comprises contacting the cleavable linker with a cleaving agent and detaching the second biomolecule-specific binding agent from the solid support;
detecting the solid support; and detecting the cleaved complex.

2. The method of claim 1, wherein the second biomolecule-specific binding agent comprises a detectable moiety.

3. The method of claim 1, wherein the cleavable linker comprises a polynucleotide or a polypeptide sequence.

4. The method of claim 1, wherein the cleavable linker comprises a polynucleotide sequence.

5. The method of claim 1, wherein cleaving the cleavable linker comprises generating an identification oligonucleotide, wherein said identification oligonucleotide comprises a portion of the polynucleotide sequence.

6. The method of claim 1, further comprising amplifying the identification oligonucleotide to generate amplification products.

7. The method of claim 6, wherein detecting the biomolecule comprises detecting the identification oligonucleotide or one or more of the amplification products.

8. The method of claim 2, wherein detecting the biomolecule comprises detecting the detectable moiety.

9. The method of claim 1, wherein the second biomolecule-specific binding agent further comprises a bioconjugate reactive moiety, an enzyme, or a label.

10. The method of claim 1, wherein the cleaving agent comprises a reducing agent, sodium periodate, or a nuclease.

11. The method of claim 1, wherein the cleaving agent comprises cleaving agent comprises RNase, Formamidopyrimidine DNA Glycosylase (Fpg), a restriction enzyme, or uracil DNA glycosylase (UDG).

12. The method of claim 1, wherein the first biomolecule-specific binding agent and the second biomolecule-specific binding agent are each independently an antibody, single-chain Fv fragment (scFv), antibody fragment-antigen binding (Fab), affimer, or an aptamer.

13. The method of claim 1, wherein the first biomolecule-specific binding agent and the second biomolecule-specific binding agent are each an antibody.

14. The method of claim 1, wherein the first biomolecule-specific binding agent is a monoclonal antibody and the second biomolecule-specific binding agent is a polyclonal antibody.

15. The method of claim 1, wherein the solid support is a particle comprising two or more fluorescent dyes.

16. The method of claim 15, wherein the two or more fluorescent dyes are selected from cyclobutenedione derivatives, symmetrical and unsymmetrical squaraines, substituted cephalosporin compounds, fluorinated squaraine compositions, alkylalkoxy squaraines, or squarylium compounds.

17. The method of claim 16, wherein the two or more fluorescent dyes are selected from 1,3-bis[(1,3-dihydro-1,3,3-trimethyl-2H-indol-2-ylidene)methyl]-2,4-dihydroxycyclobutenediylium, bis(inner salt) and 2-(3,5-dimethylpyrrol-2-yl)-4-(3,5-dimethyl-2H-pyrrol-2-ylidene)-3-hydroxy-2-cyclobuten-1-one.

18. The method of claim 1, wherein the solid support is a particle comprising polystyrene, brominated polystyrene, polyacrylic acid, polyacrylonitrile, polyacrylamide, polyacrolein, polydimethylsiloxane, polybutadiene, polyisoprene, polyurethane, polyvinyl acetate, polyvinylchloride, polyvinylpyridine, polyvinylbenzylchloride, polyvinyltoluene, polyvinylidene chloride, polydivindylbenzene, polyglycidylmethacrylate, polymethylmethacrylate, or copolymers, blends, composites, or combination thereof.

19. The method of claim 1, wherein detecting the solid support comprises detecting the two or more fluorescent dyes.

20. The method of claim 1, wherein the solid support comprises a substrate polynucleotide barcode, or the first biomolecule-specific binding agent comprises a substrate polynucleotide barcode.

21. The method of claim 20, wherein detecting the solid support comprises detecting substrate barcode.

22. A solid support, comprising:
a first biomolecule-specific binding agent attached to the solid support;
a second biomolecule-specific binding agent attached to the solid support via a cleavable linker, wherein the cleavable linker is a divalent linker comprising one or more cleavable sites, wherein the second biomolecule-specific binding agent comprises a detectable moiety.

23. The solid support of claim 22, wherein the cleavable linker is a covalent cleavable linker.

24. The solid support of claim 22, further comprising one or more oligonucleotides, wherein the one or more oligonucleotides are attached to the solid support.

25. The solid support of claim 22, wherein the solid support is a particle.

26. The solid support of claim 25, wherein said particle is in a well of a multiwell container.

27. The solid support of claim 25, wherein the average longest dimension of said particle is from about 100 nm to about 3000 nm.

28. The solid support of claim 25, wherein said particle comprises glass, ceramic, metal, silica, magnetic material, or a paramagnetic material.

29. A plurality of particles, wherein each of said particles is independently a particle of claim 25.

30. A method of amplifying a polynucleotide sequence attached to a biomolecule-specific binding agent, said method comprising:
contacting a solid support with a biomolecule, wherein said solid support comprises:
a first biomolecule-specific binding agent attached to the solid support;
a second biomolecule-specific binding agent attached to the solid support via a cleavable linker, wherein said cleavable linker comprises the polynucleotide sequence;
forming a complex comprising a biomolecule bound to both the first biomolecule-specific binding agent bound and the second specific-binding agent;
cleaving the cleavable linker of the complex thereby forming a cleaved complex; wherein cleaving comprises contacting the cleavable linker with a cleaving agent and detaching the second biomolecule-specific binding agent from the solid support; and
hybridizing a primer oligonucleotide to the polynucleotide sequence and extending the primer oligonucleotide sequence with a polymerase, thereby amplifying the polynucleotide sequence attached to the second biomolecule-specific binding agent.

31. The method of claim 30, wherein the cleavable linker is a covalent cleavable linker.


SEQUENCE LISTING (7 sequences)

SEQ ID NO: 1 - DNA, 75 bp, synthetic construct
  source 1..75: mol_type = other DNA
  modified_base order(46..48,56..58,66..68): mod_base = OTHER; note = uracil
aaaaaaaaatcgttctactacgcggtctcgttcgaccgtgcggtttttaaaaaaatttaaaaaaatttaaaaaaa

SEQ ID NO: 2 - DNA, 53 bp, synthetic construct
  source 1..53: mol_type = other DNA
  modified_base 46: mod_base = OTHER; note = uracil
aaaaaaaaatcgttctactacgcggtctcgttcgaccgtgcggtttaaaaaaa

SEQ ID NO: 3 - DNA, 55 bp, synthetic construct
  source 1..55: mol_type = other DNA
  modified_base 46..48: mod_base = OTHER; note = uracil
aaaaaaaaatcgttctactacgcggtctcgttcgaccgtgcggtttttaaaaaaa

SEQ ID NO: 4 - DNA, 57 bp, synthetic construct
  source 1..57: mol_type = other DNA
  modified_base 46..50: mod_base = OTHER; note = uracil
aaaaaaaaatcgttctactacgcggtctcgttcgaccgtgcggtttttttaaaaaaa

SEQ ID NO: 5 - DNA, 50 bp, synthetic construct
  source 1..50: mol_type = other DNA
  modified_base 46..50: mod_base = OTHER; note = uracil
aaaaaaaaatcgttctactacgcggtctcgttcgaccgtgcggttttttt

SEQ ID NO: 6 - DNA, 65 bp, synthetic construct
  source 1..65: mol_type = other DNA
  modified_base order(46..48,56..58): mod_base = OTHER; note = uracil
aaaaaaaaatcgttctactacgcggtctcgttcgaccgtgcggtttttaaaaaaatttaaaaaaa

SEQ ID NO: 7 - DNA, 72 bp, synthetic construct
  source 1..72: mol_type = other DNA
  modified_base order(46..48,63..65): mod_base = OTHER; note = uracil
aaaaaaaaatcgttctactacgcggtctcgttcgaccgtgcggtttttaaaaaaaaaaaaaatttaaaaaaa